SEQUENCE LISTING (18 sequences)

SEQ ID NO: 1 - protein, 14 aa, synthetic construct
  REGION 1..14: note = NLS of SV40 Large T-antigen
MEDPTMAPKKKRKV

SEQ ID NO: 2 - protein, 16 aa, synthetic construct
  REGION 1..16: note = NLS of nucleoplasmin
KRPAATKKAGQAKKKK

SEQ ID NO: 3 - protein, 1368 aa, Streptococcus pyogenes [sequence omitted]

SEQ ID NO: 4 - DNA, 4104 bp, synthetic construct
  misc_feature 1..4104: note = Cas9 coding sequence codon-optimized
  source 1..4104: mol_type = other DNA
atggataaaaaatatagcattggtctggatattggtaccaatagcgttggttgggcagttattaccgatgaatataaagttccgagcaaaaaatttaaagttctgggtaataccgatcgtcatagcattaaaaaaaatctgattggtgcactgctgtttgatagcggtgaaaccgcagaagcaacccgtctgaaacgtaccgcacgtcgtcgttatacccgtcgtaaaaatcgtatttgttatctgcaggaaatttttagcaatgaaatggcaaaagttgatgatagcttttttcatcgtctggaagaaagctttctggttgaagaagataaaaaacatgaacgtcatccgatttttggtaatattgttgatgaagttgcatatcatgaaaaatatccgaccatttatcatctgcgtaaaaaactggttgatagcaccgataaagcagatctgcgtctgatttatctggcactggcacatatgattaaatttcgtggtcattttctgattgaaggtgatctgaatccggataatagcgatgttgataaactgtttattcagctggttcagacctataatcagctgtttgaagaaaatccgattaatgcaagcggtgttgatgcaaaagcaattctgagcgcacgtctgagcaaaagccgtcgtctggaaaatctgattgcacagctgccgggtgaaaaaaaaaatggtctgtttggtaatctgattgcactgagcctgggtctgaccccgaattttaaaagcaattttgatctggcagaagatgcaaaactgcagctgagcaaagatacctatgatgatgatctggataatctgctggcacagattggtgatcagtatgcagatctgtttctggcagcaaaaaatctgagcgatgcaattctgctgagcgatattctgcgtgttaataccgaaattaccaaagcaccgctgagcgcaagcatgattaaacgttatgatgaacatcatcaggatctgaccctgctgaaagcactggttcgtcagcagctgccggaaaaatataaagaaattttttttgatcagagcaaaaatggttatgcaggttatattgatggtggtgcaagccaggaagaattttataaatttattaaaccgattctggaaaaaatggatggtaccgaagaactgctggttaaactgaatcgtgaagatctgctgcgtaaacagcgtacctttgataatggtagcattccgcatcagattcatctgggtgaactgcatgcaattctgcgtcgtcaggaagatttttatccgtttctgaaagataatcgtgaaaaaattgaaaaaattctgacctttcgtattccgtattatgttggtccgctggcacgtggtaatagccgttttgcatggatgacccgtaaaagcgaagaaaccattaccccgtggaattttgaagaagttgttgataaaggtgcaagcgcacagagctttattgaacgtatgaccaattttgataaaaatctgccgaatgaaaaagttctgccgaaacatagcctgctgtatgaatattttaccgtttataatgaactgaccaaagttaaatatgttaccgaaggtatgcgtaaaccggcatttctgagcggtgaacagaaaaaagcaattgttgatctgctgtttaaaaccaatcgtaaagttaccgttaaacagctgaaagaagattattttaaaaaaattgaatgttttgatagcgttgaaattagcggtgttgaagatcgttttaatgcaagcctgggtacctatcatgatctgctgaaaattattaaagataaagattttctggataatgaagaaaatgaagatattctggaagatattgttctgaccctgaccctgtttgaagatcgtgaaatgattgaagaacgtctgaaaacctatgcacatctgtttgatgataaagttatgaaacagctgaaacgtcgtcgttataccggttggggtcgtctgagccgtaaactgattaatggtattcgtgataaacagagcggtaaaaccattctggattttctgaaaagcgatggttttgcaaatcgtaattttatgcagctgattcatgatgatagcctgacctttaaagaagatattcagaaagcacaggttagcggtcagggtgatagcctgcatgaacatattgcaaatctggcaggtagcccggcaattaaaaaaggtattctgcagaccgttaaagttgttgatgaactggttaaagttatgggtcgtcataaaccggaaaatattgttattgaaatggcacgtgaaaatcagaccacccagaaaggtcagaaaaatagccgtgaacgtatgaaacgtattgaagaaggtattaaagaactgggtagccagattctgaaagaacatccggttgaaaatacccagctgcagaatgaaaaactgtatctgtattatctgcagaatggtcgtgatatgtatgttgatcaggaactggatattaatcgtctgagcgattatgatgttgatcatattgttccgcagagctttctgaaagatgatagcattgataataaagttctgacccgtagcgataaaaatcgtggtaaaagcgataatgttccgagcgaagaagttgttaaaaaaatgaaaaattattggcgtcagctgctgaatgcaaaactgattacccagcgtaaatttgataatctgaccaaagcagaacgtggtggtctgagcgaactggataaagcaggttttattaaacgtcagctggttgaaacccgtcagattaccaaacatgttgcacagattctggatagccgtatgaataccaaatatgatgaaaatgataaactgattcgtgaagttaaagttattaccctgaaaagcaaactggttagcgattttcgtaaagattttcagttttataaagttcgtgaaattaataattatcatcatgcacatgatgcatatctgaatgcagttgttggtaccgcactgattaaaaaatatccgaaactggaaagcgaatttgtttatggtgattataaagtttatgatgttcgtaaaatgattgcaaaaagcgaacaggaaattggtaaagcaaccgcaaaatattttttttatagcaatattatgaatttttttaaaaccgaaattaccctggcaaatggtgaaattcgtaaacgtccgctgattgaaaccaatggtgaaaccggtgaaattgtttgggataaaggtcgtgattttgcaaccgttcgtaaagttctgagcatgccgcaggttaatattgttaaaaaaaccgaagttcagaccggtggttttagcaaagaaagcattctgccgaaacgtaatagcgataaactgattgcacgtaaaaaagattgggatccgaaaaaatatggtggttttgatagcccgaccgttgcatatagcgttctggttgttgcaaaagttgaaaaaggtaaaagcaaaaaactgaaaagcgttaaagaactgctgggtattaccattatggaacgtagcagctttgaaaaaaatccgattgattttctggaagcaaaaggttataaagaagttaaaaaagatctgattattaaactgccgaaatatagcctgtttgaactggaaaatggtcgtaaacgtatgctggcaagcgcaggtgaactgcagaaaggtaatgaactggcactgccgagcaaatatgttaattttctgtatctggcaagccattatgaaaaactgaaaggtagcccggaagataatgaacagaaacagctgtttgttgaacagcataaacattatctggatgaaattattgaacagattagcgaatttagcaaacgtgttattctggcagatgcaaatctggataaagttctgagcgcatataataaacatcgtgataaaccgattcgtgaacaggcagaaaatattattcatctgtttaccctgaccaatctgggtgcaccggcagcatttaaatattttgataccaccattgatcgtaaacgttataccagcaccaaagaagttctggatgcaaccctgattcatcagagcattaccggtctgtatgaaacccgtattgatctgagccagctgggtggtgat

SEQ ID NO: 5 - protein, 1082 aa, synthetic construct
MKYKIGLDIGITSIGWAVINLDIPRIEDLGVRIFDRAENPKTGESLALPRRLARSARRRLRRRKHRLERIRRLFVREGILTKEELNKLFEKKHEIDVWQLRVEALDRKLNNDELARILLHLAKRRGFRSNRKSERTNKENSTMLKHIEENQSILSSYRTVAEMVVKDPKFSLHKRNKEDNYTNTVARDDLEREIKLIFAKQREYGNIVCTEAFEHEYISIWASQRPFASKDDIEKKVGFCTFEPKEKRAPKATYTFQSFTVWEHINKLRLVSPGGIRALTDDERRLIYKQAFHKNKITFHDVRTLLNLPDDTRFKGLLYDRNTTLKENEKVRFLELGAYHKIRKAIDSVYGKGAAKSFRPIDFDTFGYALTMFKDDTDIRSYLRNEYEQNGKRMENLADKVYDEELIEELLNLSFSKFGHLSLKALRNILPYMEQGEVYSTACERAGYTFTGPKKKQKTVLLPNIPPIANPVVMRALTQARKVVNAIIKKYGSPVSIHIELARELSQSFDERRKMQKEQEGNRKKNETAIRQLVEYGLTLNPTGLDIVKFKLWSEQNGKCAYSLQPIEIERLLEPGYTEVDHVIPYSRSLDDSYTNKVLVLTKENREKGNRTPAEYLGLGSERWQQFETFVLTNKQFSKKKRDRLLRLHYDENEENEFKNRNLNDTRYISRFLANFIREHLKFADSDDKQKVYTVNGRITAHLRSRWNFNKNREESNLHHAVDAAIVACTTPSDIARVTAFYQRREQNKELSKKTDPQFPQPWPHFADELQARLSKNPKESIKALNLGNYDNEKLESLQPVFVSRMPKRSITGAAHQETLRRYIGIDERSGKIQTVVKKKLSEIQLDKTGHFPMYGKESDPRTYEAIRQRLLEHNNDPKKAFQEPLYKPKKNGELGPIIRTIKIIDTTNQVIPLNDGKTVAYNSNIVRVDVFEKDGKYYCVPIYTIDMMKGILPNKAIEPNKPYSEWKEMTEDYTFRFSLYPNDLIRIEFPREKTIKTAVGEEIKIKDLFAYYQTIDSSNGGLSLVSHDNNFSLRSIGSRTLKRFEKYQVDVLGNIYKVRGEKRVGVASSSHSKAGETIRPL

SEQ ID NO: 6 - protein, 1300 aa, Francisella tularensis
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHENRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVIGTAVLEYITQQIAPKNLDNPSKKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHKLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINLLLKEKANDVHILSIDRGERHLAYYTLVDGKGNIIKQDTFNIIGNDRMKTNYHDKLAAIEKDRDSARKDWKKINNIKEMKEGYLSQVVHEIAKLVIEYNAIVVFEDLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDADANGAYHIGLKGLMLLGRIKNNQEGKKLNLVIKNEEYFEFVQNRNN

SEQ ID NO: 7 - DNA, 3900 bp, synthetic construct
  misc_feature 1..3900: note = FnCpf1
  source 1..3900: mol_type = other DNA
atgagcatttatcaggaatttgttaataaatatagcctgagcaaaaccctgcgttttgaactgattccgcagggtaaaaccctggaaaatattaaagcacgtggtctgattctggatgatgaaaaacgtgcaaaagattataaaaaagcaaaacagattattgataaatatcatcagttttttattgaagaaattctgagcagcgtttgtattagcgaagatctgctgcagaattatagcgatgtttattttaaactgaaaaaaagcgatgatgataatctgcagaaagattttaaaagcgcaaaagataccattaaaaaacagattagcgaatatattaaagatagcgaaaaatttaaaaatctgtttaatcagaatctgattgatgcaaaaaaaggtcaggaaagcgatctgattctgtggctgaaacagagcaaagataatggtattgaactgtttaaagcaaatagcgatattaccgatattgatgaagcactggaaattattaaaagctttaaaggttggaccacctattttaaaggttttcatgaaaatcgtaaaaatgtttatagcagcaatgatattccgaccagcattatttatcgtattgttgatgataatctgccgaaatttctggaaaataaagcaaaatatgaaagcctgaaagataaagcaccggaagcaattaattatgaacagattaaaaaagatctggcagaagaactgacctttgatattgattataaaaccagcgaagttaatcagcgtgtttttagcctggatgaagtttttgaaattgcaaattttaataattatctgaatcagagcggtattaccaaatttaataccattattggtggtaaatttgttaatggtgaaaataccaaacgtaaaggtattaatgaatatattaatctgtatagccagcagattaatgataaaaccctgaaaaaatataaaatgagcgttctgtttaaacagattctgagcgataccgaaagcaaaagctttgttattgataaactggaagatgatagcgatgttgttaccaccatgcagagcttttatgaacagattgcagcatttaaaaccgttgaagaaaaaagcattaaagaaaccctgagcctgctgtttgatgatctgaaagcacagaaactggatctgagcaaaatttattttaaaaatgataaaagcctgaccgatctgagccagcaggtttttgatgattatagcgttattggtaccgcagttctggaatatattacccagcagattgcaccgaaaaatctggataatccgagcaaaaaagaacaggaactgattgcaaaaaaaaccgaaaaagcaaaatatctgagcctggaaaccattaaactggcactggaagaatttaataaacatcgtgatattgataaacagtgtcgttttgaagaaattctggcaaattttgcagcaattccgatgatttttgatgaaattgcacagaataaagataatctggcacagattagcattaaatatcagaatcagggtaaaaaagatctgctgcaggcaagcgcagaagatgatgttaaagcaattaaagatctgctggatcagaccaataatctgctgcataaactgaaaatttttcatattagccagagcgaagataaagcaaatattctggataaagatgaacatttttatctggtttttgaagaatgttattttgaactggcaaatattgttccgctgtataataaaattcgtaattatattacccagaaaccgtatagcgatgaaaaatttaaactgaattttgaaaatagcaccctggcaaatggttgggataaaaataaagaaccggataataccgcaattctgtttattaaagatgataaatattatctgggtgttatgaataaaaaaaataataaaatttttgatgataaagcaattaaagaaaataaaggtgaaggttataaaaaaattgtttataaactgctgccgggtgcaaataaaatgctgccgaaagttttttttagcgcaaaaagcattaaattttataatccgagcgaagatattctgcgtattcgtaatcatagcacccataccaaaaatggtagcccgcagaaaggttatgaaaaatttgaatttaatattgaagattgtcgtaaatttattgatttttataaacagagcattagcaaacatccggaatggaaagattttggttttcgttttagcgatacccagcgttataatagcattgatgaattttatcgtgaagttgaaaatcagggttataaactgacctttgaaaatattagcgaaagctatattgatagcgttgttaatcagggtaaactgtatctgtttcagatttataataaagattttagcgcatatagcaaaggtcgtccgaatctgcataccctgtattggaaagcactgtttgatgaacgtaatctgcaggatgttgtttataaactgaatggtgaagcagaactgttttatcgtaaacagagcattccgaaaaaaattacccatccggcaaaagaagcaattgcaaataaaaataaagataatccgaaaaaagaaagcgtttttgaatatgatctgattaaagataaacgttttaccgaagataaatttttttttcattgtccgattaccattaattttaaaagcagcggtgcaaataaatttaatgatgaaattaatctgctgctgaaagaaaaagcaaatgatgttcatattctgagcattgatcgtggtgaacgtcatctggcatattataccctggttgatggtaaaggtaatattattaaacaggatacctttaatattattggtaatgatcgtatgaaaaccaattatcatgataaactggcagcaattgaaaaagatcgtgatagcgcacgtaaagattggaaaaaaattaataatattaaagaaatgaaagaaggttatctgagccaggttgttcatgaaattgcaaaactggttattgaatataatgcaattgttgtttttgaagatctgaattttggttttaaacgtggtcgttttaaagttgaaaaacaggtttatcagaaactggaaaaaatgctgattgaaaaactgaattatctggtttttaaagataatgaatttgataaaaccggtggtgttctgcgtgcatatcagctgaccgcaccgtttgaaacctttaaaaaaatgggtaaacagaccggtattatttattatgttccggcaggttttaccagcaaaatttgtccggttaccggttttgttaatcagctgtatccgaaatatgaaagcgttagcaaaagccaggaattttttagcaaatttgataaaatttgttataatctggataaaggttattttgaatttagctttgattataaaaattttggtgataaagcagcaaaaggtaaatggaccattgcaagctttggtagccgtctgattaattttcgtaatagcgataaaaatcataattgggatacccgtgaagtttatccgaccaaagaactggaaaaactgctgaaagattatagcattgaatatggtcatggtgaatgtattaaagcagcaatttgtggtgaaagcgataaaaaattttttgcaaaactgaccagcgttctgaataccattctgcagatgcgtaatagcaaaaccggtaccgaactggattatctgattagcccggttgcagatgttaatggtaatttttttgatagccgtcaggcaccgaaaaatatgccgcaggatgcagatgcaaatggtgcatatcatattggtctgaaaggtctgatgctgctgggtcgtattaaaaataatcaggaaggtaaaaaactgaatctggttattaaaaatgaagaatattttgaatttgttcagaatcgtaataat

SEQ ID NO: 8 - protein, 1263 aa, Eubacterium rectale
MNNGTNNFQNFIGISSLQKTLRNALIPTETTQQFIVKNGIIKEDELRGENRQILKDIMDDYYRGFISETLSSIDDIDWTSLFEKMEIQLKNGDNKDTLIKEQTEYRKAIHKKFANDDRFKNMFSAKLISDILPEFVIHNNNYSASEKEEKTQVIKLFSRFATSFKDYFKNRANCFSADDISSSSCHRIVNDNAEIFFSNALVYRRIVKSLSNDDINKISGDMKDSLKEMSLEEIYSYEKYGEFITQEGISFYNDICGKVNSFMNLYCQKNKENKNLYKLQKLHKQILCIADTSYEVPYKFESDEEVYQSVNGFLDNISSKHIVERLRKIGDNYNGYNLDKIYIVSKFYESVSQKTYRDWETINTALEIHYNNILPGNGKSKADKVKKAVKNDLQKSITEINELVSNYKLCSDDNIKAETYIHEISHILNNFEAQELKYNPEIHLVESELKASELKNVLDVIMNAFHWCSVFMTEELVDKDNNFYAELEEIYDEIYPVISLYNLVRNYVTQKPYSTKKIKLNFGIPTLADGWSKSKEYSNNAIILMRDNLYYLGIFNAKNKPDKKIIEGNTSENKGDYKKMIYNLLPGPNKMIPKVFLSSKTGVETYKPSAYILEGYKQNKHIKSSKDFDITFCHDLIDYFKNCIAIHPEWKNFGFDFSDTSTYEDISGFYREVELQGYKIDWTYISEKDIDLLQEKGQLYLFQIYNKDFSKKSTGNDNLHTMYLKNLFSEENLKDIVLKLNGEAEIFFRKSSIKNPIIHKKGSILVNRTYEAEEKDQFGNIQIVRKNIPENIYQELYKYFNDKSDKELSDEAAKLKNVVGHHEAATNIVKDYRYTYDKYFLHMPITINFKANKTGFINDRILQYIAKEKDLHVIGIDRGERNLIYVSVIDTCGNIVEQKSFNIVNGYDYQIKLKQQEGARQIARKEWKEIGKIKEIKEGYLSLVIHEISKMVIKYNAIIAMEDLSYGFKKGRFKVERQVYQKFETMLINKLNYLVFKDISITENGGLLKGYQLTYIPDKLKNVGHQCGCIFYVPAAYTSKIDPTTGFVNIFKFKDLTVDAKREFIKKFDSIRYDSEKNLFCFTFDYNNFITQNTVMSKSSWSVYTYGVRIKRRFVNGRFSNESDTIDITKDMEKTLEMTDINWRDGHDLRQDIIDYEIVQHIFEIFRLTVQMRNSLSELEDRDYDRLISPVLNENNIFYDSAKAGDALPKDADANGAYCIALKGLYEIKQITENWKEDGKFSRDKLKISNKDWFDFIQNKRYL

SEQ ID NO: 9 - protein, 1274 aa, synthetic construct
  REGION 1..1274: note = Mad7-NLS
MNNGTNNFQNFIGISSLQKTLRNALIPTETTQQFIVKNGIIKEDELRGENRQILKDIMDDYYRGFISETLSSIDDIDWTSLFEKMEIQLKNGDNKDTLIKEQTEYRKAIHKKFANDDRFKNMFSAKLISDILPEFVIHNNNYSASEKEEKTQVIKLFSRFATSFKDYFKNRANCFSADDISSSSCHRIVNDNAEIFFSNALVYRRIVKSLSNDDINKISGDMKDSLKEMSLEEIYSYEKYGEFITQEGISFYNDICGKVNSFMNLYCQKNKENKNLYKLQKLHKQILCIADTSYEVPYKFESDEEVYQSVNGFLDNISSKHIVERLRKIGDNYNGYNLDKIYIVSKFYESVSQKTYRDWETINTALEIHYNNILPGNGKSKADKVKKAVKNDLQKSITEINELVSNYKLCSDDNIKAETYIHEISHILNNFEAQELKYNPEIHLVESELKASELKNVLDVIMNAFHWCSVFMTEELVDKDNNFYAELEEIYDEIYPVISLYNLVRNYVTQKPYSTKKIKLNFGIPTLADGWSKSKEYSNNAIILMRDNLYYLGIFNAKNKPDKKIIEGNTSENKGDYKKMIYNLLPGPNKMIPKVFLSSKTGVETYKPSAYILEGYKQNKHIKSSKDFDITFCHDLIDYFKNCIAIHPEWKNFGFDFSDTSTYEDISGFYREVELQGYKIDWTYISEKDIDLLQEKGQLYLFQIYNKDFSKKSTGNDNLHTMYLKNLFSEENLKDIVLKLNGEAEIFFRKSSIKNPIIHKKGSILVNRTYEAEEKDQFGNIQIVRKNIPENIYQELYKYFNDKSDKELSDEAAKLKNVVGHHEAATNIVKDYRYTYDKYFLHMPITINFKANKTGFINDRILQYIAKEKDLHVIGIDRGERNLIYVSVIDTCGNIVEQKSFNIVNGYDYQIKLKQQEGARQIARKEWKEIGKIKEIKEGYLSLVIHEISKMVIKYNAIIAMEDLSYGFKKGRFKVERQVYQKFETMLINKLNYLVFKDISITENGGLLKGYQLTYIPDKLKNVGHQCGCIFYVPAAYTSKIDPTTGFVNIFKFKDLTVDAKREFIKKFDSIRYDSEKNLFCFTFDYNNFITQNTVMSKSSWSVYTYGVRIKRRFVNGRFSNESDTIDITKDMEKTLEMTDINWRDGHDLRQDIIDYEIVQHIFEIFRLTVQMRNSLSELEDRDYDRLISPVLNENNIFYDSAKAGDALPKDADANGAYCIALKGLYEIKQITENWKEDGKFSRDKLKISNKDWFDFIQNKRYLSGGSPKKKRKV

SEQ ID NO: 10 - DNA, 22 bp, synthetic construct
  misc_feature 1..22: note = forward primer
  source 1..22: mol_type = other DNA
cagacacaatggtagatgatgg

SEQ ID NO: 11 - DNA, 21 bp, synthetic construct
  misc_feature 1..21: note = reversed primer
  source 1..21: mol_type = other DNA
cttcatcgccatgcccagaag

SEQ ID NO: 12 - DNA, 20 bp, synthetic construct
  misc_feature 1..20: note = forward primer
  source 1..20: mol_type = other DNA
taagaaaccgaagcaaactc

SEQ ID NO: 13 - DNA, 20 bp, synthetic construct
  misc_feature 1..20: note = reversed primer
  source 1..20: mol_type = other DNA
gcgctttctacaatcttaca

SEQ ID NO: 14 - DNA, 24 bp, synthetic construct
  misc_feature 1..24: note = forward primer
  source 1..24: mol_type = other DNA
atactaatttccacccaacaacgt

SEQ ID NO: 15 - DNA, 24 bp, synthetic construct
  misc_feature 1..24: note = reversed primer
  source 1..24: mol_type = other DNA
tctcaacattaaacatgtcgccac

SEQ ID NO: 16 - protein, 182 aa, Arabidopsis thaliana
MSFSVKGRSLRGNNNGGTGTKCGRWNPTVEQLKILTDLFRAGLRTPTTDQIQKISTELSFYGKIESKNVFYWFQNHKARERQKRRKISIDFDHHHHQPSTRDVFEISEEDCQEEEKVIETLQLFPVNSFEDSNSKVDKMRARGNNQYREYIRETTTTSFSPYSSCGAEMEHPPPLDLRLSFL

SEQ ID NO: 17 - protein, 574 aa, Arabidopsis thaliana
MNSNNWLGFPLSPNNSSLPPHEYNLGLVSDHMDNPFQTQEWNMINPHGGGGDEGGEVPKVADFLGVSKPDENQSNHLVAYNDSDYYFHTNSLMPSVQSNDVVVAACDSNTPNNSSYHELQESAHNLQSLTLSMGTTAGNNVVDKASPSETTGDNASGGALAVVETATPRRALDTFGQRTSIYRGVTRHRWTGRYEAHLWDNSCRREGQSRKGRQVYLGGYDKEDKAARSYDLAALKYWGPSTTTNFPITNYEKEVEEMKHMTRQEFVAAIRRKSSGFSRGASMYRGVTRHHQHGRWQARIGRVAGNKDLYLGTFSTEEEAAEAYDIAAIKFRGLNAVTNFEINRYDVKAILESSTLPIGGGAAKRLKEAQALESSRKREAEMIALGSSFQYGGGSSTGSGSTSSRLQLQPYPLSIQQPLEPFLSLQNNDISHYNNNNAHDSSSFNHHSYIQTQLHLHQQTNNYLQQQSSQNSQQLYNAYLHSNPALLHGLVSTSIVDNNNNNGGSSGSYNTAAFLGNHGIGIGSSSTVGSTEEFPTVKTDYDMPSSDGTGGYSGWTSESVQGSNPGGVFTMWNE

SEQ ID NO: 18 - protein, 334 aa, Arabidopsis thaliana
MAAAMNLYTCSRSFQDSGGELMDALVPFIKSVSDSPSSSSAASASAFLHPSAFSLPPLPGYYPDSTFLTQPFSYGSDLQQTGSLIGLNNLSSSQIHQIQSQIHHPLPPTHHNNNNSFSNLLSPKPLLMKQSGVAGSCFAYGSGVPSKPTKLYRGVRQRHWGKWVAEIRLPRNRTRLWLGTFDTAEEAALAYDKAAYKLRGDFARLNFPNLRHNGSHIGGDFGEYKPLHSSVDAKLEAICKSMAETQKQDKSTKSSKKREKKVSSPDLSEKVKAEENSVSIGGSPPVTEFEESTAGSSPLSDLTFADPEEPPQWNETFSLEKYPSYEIDWDSILA

(19) United States
(12) Patent Application Publication
(10) Pub. No.: US 2024/0175040 A1
(43) Pub. Date: May 30, 2024

(54) CO-REGENERATION RECALCITRANT PLANTS

(71) Applicant: Keygene N.V., Wageningen (NL)

(72) Inventors: Jeroen STUURMAN, Wageningen (NL); Bipna Rani SHRESTHA, Wageningen (NL); Hugo Ferdinand HOFHUIS, Wageningen (NL)

(73) Assignee: Keygene N.V., Wageningen (NL)

(21) Appl. No.: 18/487,029

(22) Filed: Oct. 13, 2023

Related U.S. Application Data
(63) Continuation of application No. PCT/EP22/60173, filed on Apr. 15, 2022.

(30) Foreign Application Priority Data
Apr. 15, 2021 (EP) 21168630.8

(51) Int. Cl. C12N 15/82; C12N 9/22

(52) U.S. Cl. CPC C12N 15/8202; C12N 9/22; C12N 2310/20

(57) ABSTRACT

The invention concerns a method for producing a shoot of a plant comprising germline progenitor cells of a recalcitrant plant. The germline progenitor cells may be modified to comprise a mutation in a sequence of interest. The invention further pertains to plants obtainable by the method of the invention, wherein the plant preferably comprises at least the L2-meristem layer of the recalcitrant plant.

### CROSS-REFERENCE TO RELATED APPLICATIONS

This application is a continuation of International Patent Application No. PCT/EP2022/060173 filed Apr. 15, 2022, which application claims priority to European Patent Application No. 21168630.8 filed Apr. 15, 2021, the contents of which are all incorporated herein by reference in their entireties.

### SEQUENCE LISTING

The present application is filed along with a Sequence Listing in electronic format. The Sequence Listing is provided as a file entitled 085342-4600.xml, created on Feb. 20, 2024, which is 36,207 bytes in size. The information in the electronic format of the Sequence Listing is incorporated herein by reference in its entirety.

### FIELD OF THE INVENTION

The present invention relates to the field of molecular plant biology, in particular to the field of plant regeneration. The invention concerns methods for improving the regeneration capacity and/or efficiency of plants.

### BACKGROUND

Many workflows in plant biotechnology require regeneration from single cells to whole plants. As in general plant cells have a limited capacity to regenerate, the regeneration step is often the major bottleneck of such workflows. Regeneration potential is highly dependent on plant species, plant variety and plant tissue origin. Even with established protocols, the fraction of cells successfully regenerating to plants is usually quite low (Srinivasan et al., Planta 2007, 225:341-351). Plant species or varieties in which the regeneration fails or the efficiency is poor are considered recalcitrant.

Non-limiting examples of workflows for which regeneration is a bottleneck are the general multiplication of (clonally propagated) plant material, especially in case of haploid plant material or genetically complex (e.g. highly ploidy and heterozygous) F1 populations, but also more advanced plant biotech workflows such as, but not limited to, targeted plant genome editing, production of (stable or transient) transformants and doubled haploid induction.

Genome editing (GE), by CRISPR/Cas9 or related technologies, ranks among a handful of major breakthroughs in agricultural biotechnology which has the potential to easily convert improved traits to crops by making quick and efficient directed and heritable mutations in plants. It is to be expected that this technology will gain momentum over the next decade, and will be of large influence on research and development of crop improvement and breeding. To obtain a plant carrying a heritable mutation, the mutation can be introduced in a plant protoplast, followed by regeneration of the protoplast into a plant. A major hurdle of this technology is the inability and/or genotype dependence of many crop species to regenerate whole plants from single edited cells. The latter is a key unresolved factor, a.o. for applied GE as it moves towards DNA-free systems (Zhang et al. 2021 Plant Communications 2 (100168) p1-p13) that mostly rely on delivery to protoplasts.

An alternative approach is the inoculation of a plant with a vector that induces a heritable mutation, such as viral vectors. The main limitation to this approach is that in many systems, viruses are actively excluded from the shoot apical meristem (SAM) and silenced by a strong post-transcriptional gene silencing mechanism. Only somatic edits can therefore be obtained that typically are not directly transmissible to the progeny, since generative tissues develop from meristems. Therefore, also this process therefore benefits from an improved regeneration protocol. There is thus a strong need in the art to increase the regeneration efficiency of a plant, preferably to increase the regeneration efficiency of a recalcitrant plant. In particular, there is a strong need for increasing the regeneration efficiency of a plant carrying a heritable mutation.

### SUMMARY

The invention may be summarized in the following embodiments:

Embodiment 1. Method of generating and selecting a shoot of a plant, wherein the method comprises the steps of:
(a) contacting a cell of a recalcitrant plant with a cell of a regenerative plant, wherein the cell of the regenerative plant shows a higher regeneration efficiency than the cell of the recalcitrant plant under a condition that allows for shoot regeneration;
(b) allowing the contacted cells of step (a) to form one or more shoots under the condition that allows for shoot regeneration;
(c) selecting a shoot formed in step (b), wherein at least part of said shoot consists of cells of the recalcitrant plant; and optionally
(d) growing a plant from the selected shoot of step (c).

Embodiment 2. Method according to embodiment 1, wherein in step (c) the part of the selected shoot that consists of cells of the recalcitrant plant is a tissue comprising germline progenitor cells, and wherein optionally the method further comprises step (d) and a step of obtaining seed or plant progeny of the plant grown in step (d) by sexual propagation, optionally by selfing or backcrossing.

Embodiment 3. Method according to embodiment 1 or 2, wherein in step (c) the selected shoot consists of cells of the recalcitrant plant, and wherein optionally the method further comprises step (d) and a step of obtaining progeny of the plant grown in step (d) by vegetative propagation.

Embodiment 4. Method according to embodiments 1 to 3, wherein the cell of the recalcitrant plant and the cell of the regenerative plant of step (a) are isolated cells, preferably protoplasts.

Embodiment 5. Method according to embodiment 4, wherein the isolated cells in step a) are exposed to a compound promoting aggregation of the cell membranes of the cells, preferably by using a plant cell and/or protoplast linking agent, and wherein preferably the linking agent is Yariv reagent.

Embodiment 6. Method according to embodiments 1 to 3, wherein the cell of the recalcitrant plant and the cell of the regenerative plant of step (a) are comprised in a tissue.

Embodiment 7. Method according to embodiment 6, wherein step (a) is performed by stock-scion grafting and allowing the graft junction to heal.

Embodiment 8. Method according to embodiment 7, wherein step (b) comprises the steps of:
generating a wound at or near the graft junction;
allowing callus to be formed at the wounded graft junction; and
allowing a shoot to grow from said callus.

Embodiment 9. Method according to any one of the preceding embodiments, wherein the method further comprises a step of introducing in the cell of the recalcitrant plant of step
(a) or in a cell originating therefrom in the shoot formed in step (b):
   (i) a transgene; or
   (ii) a mutation in a sequence of interest.

Embodiment 10. Method according to embodiment 9, wherein the step of introducing the transgene or the mutation is prior to step (b), and optionally prior to step (a).

Embodiment 11. Method according to embodiment 9 or 10, wherein the transgene or the mutation is comprised in at least one of:
the germline progenitor cell and/or a germline cell derived therefrom; and
a plant part of the plant grown in step (d) used for vegetative propagation, of the shoot formed in step (b).

Embodiment 12. Method according to any one of embodiments 9-11, wherein the mutation is introduced by programmed genome editing, preferably using a site-specific endonuclease, preferably a CRISPR endonuclease.

Embodiment 13. Plant obtainable from the method of any one of the embodiments 9-12, wherein said plant comprises at least one of
i) a germline progenitor cell and/or a germline cell derived therefrom, of the recalcitrant plant; and
ii) a plant part for clonal propagation of the recalcitrant plant,
wherein the germline progenitor cell, germline cell and/or plant part comprises the transgene or the mutation in the sequence of interest.

Embodiment 14. Plant according to embodiment 13, wherein said plant comprises cells of the recalcitrant plant and cells of the regenerative plant.

Embodiment 15. Use of an agent linking a cell membrane of a cell of a recalcitrant plant to a cell of a regenerative plant for regeneration of said recalcitrant plant cell, wherein preferably said reagent is Yariv reagent.

### Definitions

Various terms relating to the methods, compositions, uses and other aspects of the present invention are used throughout the specification and claims. Such terms are to be given their ordinary meaning in the art to which the invention pertains, unless otherwise indicated. Other specifically defined terms are to be construed in a manner consistent with the definition provided herein.

It is clear for the skilled person that any methods and materials similar or equivalent to those described herein can be used for practising the present invention.

Methods of carrying out the conventional techniques used in methods of the invention will be evident to the skilled worker. The practice of conventional techniques in molecular biology, biochemistry, computational chemistry, cell culture, recombinant DNA, bioinformatics, genomics, sequencing and related fields are well-known to those of skill in the art and are discussed, for example, in the following literature references: Sambrook et al. Molecular Cloning. A Laboratory Manual, 2nd Edition, Cold Spring Harbor Laboratory Press, Cold Spring Harbor, N. Y., 1989; Ausubel et al.. Current Protocols in Molecular Biology, John Wiley & Sons, New York, 1987 and periodic updates; and the series Methods in Enzymology, Academic Press, San Diego.

The singular terms “a,” “an,” and “the” include plural referents unless the context clearly dictates otherwise. Thus, for example, reference to “a cell” includes a combination of two or more cells, and the like. The indefinite article “a” or “an” thus usually means “at least one”.

The term “and/or” refers to a situation wherein one or more of the stated cases may occur, alone or in combination with at least one of the stated cases, up to with all of the stated cases.

As used herein, the term “about” is used to describe and account for small variations. For example, the term can refer to less than or equal to ±(+or −) 10%, such as less than or equal to±5%, less than or equal to±4%, less than or equal to±3%, less than or equal to±2%, less than or equal to±1%, less than or equal to±0.5%, less than or equal to±0.1%, or less than or equal to±0.05%. Additionally, amounts, ratios, and other numerical values are sometimes presented herein in a range format. It is to be understood that such range format is used for convenience and brevity and should be understood flexibly to include numerical values explicitly specified as limits of a range, but also to include all individual numerical values or sub-ranges encompassed within that range as if each numerical value and sub-range is explicitly specified. For example, a ratio in the range of about 1 to about 200 should be understood to include the explicitly recited limits of about 1 and about 200, but also to include individual ratios such as about 2, about 3, and about 4, and sub-ranges such as about 10 to about 50, about 20 to about 100, and so forth.

The term “comprising” is construed as being inclusive and open ended, and not exclusive. Specifically, the term and variations thereof mean the specified features, steps or components are included. These terms are not to be interpreted to exclude the presence of other features, steps or components.

The terms “protein” or “polypeptide” are used interchangeably and refer to molecules consisting of a chain of amino acids, without reference to a specific mode of action, size, 3 dimensional structure or origin. A “fragment” or “portion” of a protein may thus still be referred to as a “protein”. An “isolated protein” is used to refer to a protein which is no longer in its natural environment, for example in vitro or in a recombinant bacterial or plant host cell.

“Plant” refers to either the whole plant or to parts of a plant tissue or organs (e.g. pollen, seeds, roots, leaves, flowers, flower buds, anthers, fruit, etc.) obtainable from the plant, as well as derivatives of any of these and progeny derived from such a plant by selfing or crossing or apomictic reproduction. Non-limiting examples of plants include crop plants and cultivated plants, such as Affrican eggplant, alliums, artichoke, asparagus, barley, beet, bell pepper, bitter gourd, bladder cherry, bottle gourd, cabbage, canola, carrot, cassava, cauliflower, celery, chicory, common bean, corn salad, cotton, cucumber, eggplant, endive, fennel, gherkin, grape, hot pepper, lettuce, maize, melon, oilseed rape, okra, parsley, parsnip, pepino, pepper, potato, pumpkin, radish, rice, ridge gourd, rocket, rye, snake gourd, sorghum, spinach, sponge gourd, squash, sugar beet, sugar cane, sunflower, tomatillo, tomato, tomato rootstock, vegetable Brassica, watermelon, wax gourd, wheat and zucchini.

“Plant cell(s)” include protoplasts, gametes, suspension cultures, microspores, pollen grains, etc., either in isolation or within a tissue, organ or organism, from plant origin. The plant cell can e.g. be part of a multicellular structure, such as a callus, meristem, plant organ or an explant. A plant cell may be a meristematic cell, a somatic cell and/or a reproductive cell.

“Similar conditions” for culturing the plant/plant cells means among other things the use of a similar temperature, humidity, nutrition and light conditions, and similar irrigation and day/night rhythm.

The terms “homology”, “sequence identity” and the like are used interchangeably herein. Sequence identity is herein defined as a relationship between two or more amino acid (polypeptide or protein) sequences or two or more nucleotide (polynucleotide) sequences, as determined by comparing the sequences. In the art, “identity” also means the degree of sequence relatedness between amino acid or nucleic acid sequences, as the case may be, as determined by the match between strings of such sequences. “Similarity” between two amino acid sequences is determined by comparing the amino acid sequence and its conserved amino acid substitutes of one polypeptide to the sequence of a second polypeptide. “Identity” and “similarity” can be readily calculated by known methods. The percentage sequence identity/similarity can be determined over the full length of the sequence.

As used herein “sequence identity” refers to the extent to which two optimally aligned polynucleotide or peptide sequences are invariant throughout a window of alignment of components, e.g., nucleotides or amino acids. An “identity fraction” for aligned segments of a test sequence and a reference sequence is the number of identical components which are shared by the two aligned sequences divided by the total number of components in reference sequence segment, i.e., the entire reference sequence or a smaller defined part of the reference sequence. “Percent identity” is the identity fraction times 100.

“Sequence identity” and “sequence similarity” can be determined by alignment of two peptide or two nucleotide sequences using global or local alignment algorithms, depending on the length of the two sequences. Sequences of similar lengths are preferably aligned using a global alignment algorithms (e.g. Needleman Wunsch) which aligns the sequences optimally over the entire length, while sequences of substantially different lengths are preferably aligned using a local alignment algorithm (e.g. Smith Waterman). Sequences may then be referred to as “substantially identical” or “essentially similar” when they (when optimally aligned by for example the programs GAP or BESTFIT using default parameters) share at least a certain minimal percentage of sequence identity (as defined herein). The percent of sequence identity is preferably determined using the “BESTFIT” or “GAP” program of the Sequence Analysis Software Package™ (Version 10; Genetics Computer Group, Inc., Madison, Wis.). GAP uses the Needleman and Wunsch global alignment algorithm (Needleman and Wunsch, Journal of Molecular Biology 48:443-453, 1970) to align two sequences over their entire length (full length), maximizing the number of matches and minimizing the number of gaps. A global alignment is suitably used to determine sequence identity when the two sequences have similar lengths. Generally, the GAP default parameters are used, with a gap creation penalty=50 (nucleotides)/8 (proteins) and gap extension penalty=3 (nucleotides)/2 (proteins). For nucleotides the default scoring matrix used is nwsgapdna and for proteins the default scoring matrix is Blosum62 (Henikoff & Henikoff, 1992, PNAS 89, 915-919). Sequence alignments and scores for percentage sequence identity may be determined using computer programs, such as the GCG Wisconsin Package, Version 10.3, available from Accelrys Inc., 9685 Scranton Road, San Diego, CA 92121-3752 USA, or using open source software, such as the program “needle” (using the global Needleman Wunsch algorithm) or “water” (using the local Smith Waterman algorithm) in EmbossWIN version 2.10.0, using the same parameters as for GAP above, or using the default settings (both for ‘needle’ and for ‘water’ and both for protein and for DNA alignments, the default Gap opening penalty is 10.0 and the default gap extension penalty is 0.5; default scoring matrices are Blossum62 for proteins and DNAFull for DNA). gap extension penalty is 0.5; default scoring matrices are Blossum62 for proteins and DNAFull for DNA). “BESTFIT” performs an optimal alignment of the best segment of similarity between two sequences and inserts gaps to maximize the number of matches using the local homology algorithm of Smith and Waterman (Smith and Waterman, Advances in Applied Mathematics, 2:482-489, 1981, Smith et al., Nucleic Acids Research 11:2205-2220, 1983). When sequences have a substantially different overall lengths, local alignments, such as those using the Smith Waterman algorithm, are preferred.

Useful methods for determining sequence identity are also disclosed in Guide to Huge Computers, Martin J. Bishop, ed., Academic Press, San Diego, 1994, and Carillo, H., and Lipton, D., Applied Math (1988) 48:1073. More particularly, preferred computer programs for determining sequence identity include the Basic Local Alignment Search Tool (BLAST) programs which are publicly available from National Center Biotechnology Information (NCBI) at the National Library of Medicine, National Institute of Health, Bethesda, Md. 20894; see BLAST Manual, Altschul et al., NCBI, NLM, NIH; Altschul et al., J. Mol. Biol. 215:403-410 (1990); version 2.0 or higher of BLAST programs allows the introduction of gaps (deletions and insertions) into alignments; for peptide sequence BLASTX can be used to determine sequence identity; and, for polynucleotide sequence BLASTN can be used to determine sequence identity.

Alternatively percentage similarity or identity may be determined by searching against public databases, using algorithms such as FASTA, BLAST, etc. Thus, the nucleic acid and protein sequences described herein can further be used as a “query sequence” to perform a search against public databases to, for example, identify other family members or related sequences. Such searches can be performed using the BLASTn and BLASTx programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to nucleic acid molecules described herein. BLAST protein searches can be performed with the BLASTx program, score=50, wordlength=3 to obtain amino acid sequences homologous to protein molecules described herein. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., BLASTx and BLASTn) can be used. See the homepage of the National Center for Biotechnology Information at http://www.ncbi.nlm.nih.gov/.

A “nucleic acid” or “polynucleotide” as used herein may include any polymer or oligomer of pyrimidine and purine bases, preferably cytosine, thymine, and uracil, and adenine and guanine, respectively (See Albert L. Lehninger, Principles of Biochemistry, at 793-800 (Worth Pub. 1982) which is herein incorporated by reference in its entirety for all purposes). Contemplated are any deoxyribonucleotide, ribonucleotide or nucleic acid component, and any chemical variants thereof, such as methylated, hydroxymethylated or glycosylated forms of these bases, and the like. The polymers or oligomers may be heterogeneous or homogenous in composition, and may be isolated from naturally occurring sources or may be artificially or synthetically produced. In addition, the nucleic acids may be DNA (optionally cDNA) or RNA, or a mixture thereof, and may exist permanently or transitionally in single-stranded or double-stranded form, including homoduplex, heteroduplex, and hybrid states.

An “isolated nucleic acid” is used to refer to a nucleic acid which is no longer in its natural environment, for example in vitro or in a recombinant bacterial or plant cell. A nucleic acid and/or protein may be at least one of a recombinant, synthetic or artificial nucleic acid and/or protein.

The terms “nucleic acid construct”, “nucleic acid vector”, “vector” and “expression construct” are used interchangeably herein and is herein defined as a man-made nucleic acid molecule resulting from the use of recombinant DNA technology. The terms “nucleic acid construct” and “nucleic acid vector” therefore does not include naturally occurring nucleic acid molecules although a nucleic acid construct may comprise (parts of) naturally occurring nucleic acid molecules.

The vector backbone may for example be a binary or superbinary vector (see e.g. U.S. Pat. No. 5,591,616, US 2002138879 and WO 95/06722), a co-integrate vector or a T-DNA vector, as known in the art and as described elsewhere herein, into which a chimeric gene is integrated or, if a suitable transcription regulatory sequence is already present, only a desired nucleic acid sequence (e.g. a coding sequence, an antisense or an inverted repeat sequence) is integrated downstream of the transcription regulatory sequence. Vectors can comprise further genetic elements to facilitate their use in molecular cloning, such as e.g. selectable markers, multiple cloning sites and the like.

The term “gene” means a DNA fragment comprising a region (transcribed region), which is transcribed into an RNA molecule (e.g. an mRNA) in a cell, operably linked to suitable regulatory regions (e.g. a promoter). A gene will usually comprise several operably linked fragments, such as a promoter, a 5′ leader sequence, a coding region and a 3′ non-translated sequence (3′ end) comprising a polyadenylation site.

“Expression of a gene” refers to the process wherein a DNA region which is operably linked to appropriate regulatory regions, particularly a promoter, is transcribed into an RNA, which is biologically active, e.g. which is capable of being translated into a biologically active protein or peptide, or e.g. a regulatory non-coding RNA.

The term “operably linked” refers to a linkage of polynucleotide elements in a functional relationship. A nucleic acid is “operably linked” when it is placed into a functional relationship with another nucleic acid sequence. For instance, a promoter, or rather a transcription regulatory sequence, is operably linked to a coding sequence if it affects the transcription of the coding sequence. Operably linked may mean that the DNA sequences being linked are contiguous.

“Promoter” refers to a nucleic acid fragment that functions to control the transcription of one or more nucleic acids. A promoter fragment is preferably located upstream (5′) with respect to the direction of transcription of the transcription initiation site of the gene, and is structurally identified by the presence of a binding site for DNA-dependent RNA polymerase, transcription initiation site(s) and can further comprise any other DNA sequences, including, but not limited to transcription factor binding sites, repressor and activator protein binding sites, and any other sequences of nucleotides known to one of skill in the art to act directly or indirectly to regulate the amount of transcription from the promoter.

A “constitutive” promoter is a promoter that is active in most tissues under most physiological and developmental conditions. An “inducible” promoter is a promoter that is physiologically (e.g. by external application of certain compounds) or developmentally regulated. A “tissue specific” promoter is only active in specific types of tissues or cells.

Optionally the term “promoter” may also include the 5′ UTR region (5′ Untranslated Region) (e.g. the promoter may herein include one or more parts upstream of the translation initiation codon of transcribed region, as this region may have a role in regulating transcription and/or translation).

A “3′ UTR” or “3′ non-translated sequence” (also often referred to as 3′ untranslated region, or 3′end) refers to the nucleic acid sequence found downstream of the coding sequence of a gene, which comprises for example a transcription termination site and (in most, but not all eukaryotic mRNAs) a polyadenylation signal (such as e.g. AAUAAA or variants thereof). After termination of transcription, the mRNA transcript may be cleaved downstream of the polyadenylation signal and a poly(A) tail may be added, which is involved in the transport of the mRNA to the cytoplasm (where translation takes place).

The term “cDNA” means complementary DNA. Complementary DNA is made by reverse transcribing RNA into a complementary DNA sequence. cDNA sequences thus correspond to RNA sequences that are expressed from genes. As RNA sequences expressed from the genome can undergo splicing, i.e. introns are spliced out of the pre-mRNA and exons are joined together, before being translated in the cytoplasm into proteins, it is understood that expression of a cDNA means expression of the mRNA that encodes for the cDNA. The cDNA sequence thus may not be identical to the genomic DNA sequence to which it corresponds as the cDNA may encode only the complete open reading frame, consisting of the joined exons, for a protein, whereas the genomic DNA sequence may comprise exon sequences interspersed by intron sequences. Genetically modifying a gene which encodes a protein may thus not only relate to modifying the sequences encoding the protein, but may also involve mutating intronic sequences of the genomic DNA and/or other gene regulatory sequences of that gene.

The term “regeneration” is herein defined as the formation of a new tissue and/or a new organ from a single plant cell, a group of cells, a callus, an explant, a tissue or from an organ. Regeneration may include the formation of a new plant from a single plant cell or from e.g. a callus, an explant, a tissue or an organ. The plant cell for regeneration can be an undifferentiated plant cell. A preferred plant cell is a protoplast. The regeneration process can occur directly from parental tissues or indirectly, e.g. via the formation of a callus. The regeneration pathway can be somatic embryogenesis or organogenesis. Somatic embryogenesis is understood herein as the formation of somatic embryos, which can be grown into whole plants. Organogenesis is understood herein as the formation of new organs from (undifferentiated) cells. Organogenesis may be at least one of meristem formation, adventitious shoot formation, inflorescence formation, root formation, elongation of adventitious shoots and (subsequent) the formation of a complete plant. Preferably, regeneration is at least one of shoot regeneration, (ectopic) apical meristem formation and root regeneration. Shoot regeneration as defined herein is de novo shoot formation. For example, regeneration can be the regeneration of a(n) (inflorescence) shoot from a(n) (elongated) hypocotyl explant.

The term “normal growth conditions” is herein understood as an environment wherein a plant grows. Such conditions include at minimum a suitable temperature (i.e. between 0° C.-60° C.), nutrition, day/night rhythm and irrigation.

The term “conditions that allow for regeneration” is herein understood as an environment wherein a plant cell or tissue can regenerate, preferably including normal growth conditions.

“Shoot organogenesis” is the regeneration pathway by which cells, preferably cells of callus or explant, form a de novo shoot apical meristem that develops into a shoot with leaf primordia and leaves. As there is only one apical meristem, this is a unipolar structure, and roots are not formed at this stage. The vascular system of the shoot is often connected to the parent tissue. Only after the shoots have fully formed and elongated, and are taken off e.g. the callus or explant, can the formation of roots be induced in a separate root induction step on a different culture medium (Thorpe, TA (1993) In vitro Organogenesis and Somatic Embryogenesis: Physiological and Biochemical Aspects. In: Roubelakis-Angelakis K. A., Van Thanh K. T. (eds) Morphogenesis in Plants. NATO ASI Series (Series A: Life Sciences), Vol. 253. Springer, Boston, MA).

Shoot organogenesis may occur spontaneously, i.e. without the external addition of any plant growth regulators (PGRs). Shoot organogenesis may be induced by plant growth regulators, usually cytokinins alone in different concentrations or in combination with an auxin, wherein preferably the cytokinins remain a constituent of the culture media until the new shoot apical meristems and the shoots have been formed and are sufficiently elongated, e.g. to take them off the primary explant or callus. Preferably, for the induction of shoot formation, the concentration of cytokinins exceeds the concentration of auxins.

“Somatic embryogenesis” leads to the formation of bipolar structures resembling zygotic embryos, which contain a root-shoot axis with a closed independent vascular system. In other words, both root and shoot primordia are being formed simultaneously, and there is no vascular connection to the underlying tissue (Dodds, J H and Roberts, L W (1985) Experiments in plant tissue culture. Cambridge University Press, Cambridge, UK). Somatic embryogenesis can e.g. be induced indirectly from callus or cell suspensions, or they can be induced directly on cells of explants (Thorpe, supra). Somatic embryo formation passes through a number of distinct stages, from globular stage (small isodiametric cell clusters), via heart stage (bilaterally symmetrical structures) to torpedo stage (elongation). The globular-to-heart transition is marked by the outgrowth of the two cotyledons and the beginning of the development of the radicle (Zimmerman, J L (1993) Somatic Embryogenesis: A Model for Early Development in Higher Plants. The Plant Cell 5:1411-1423; Von Arnold et al (2002) Developmental pathways of somatic embryogenesis. Plant Cell, Tissue and Organ Culture 69:233-249). Finally, torpedo-stage somatic embryos can develop into plantlets that contain green cotyledons, elongated hypocotyls, and developed radicles with clearly differentiated root hairs (Zimmerman, supra), in a process that is termed ‘germination’ (analogous to zygotic embryos) or ‘conversion’ or ‘maturation’ (Von Arnold et al., supra). In the induction of somatic embryogenesis, directly or indirectly, preferably auxins are used at the initial stage to induce an embryogenic state in the callus, but the embryos form after passage of the culture to a medium without or with reduced auxin levels. Auxins used for somatic embryo induction are e.g. 1-naphthaleneacetic acid (NAA), 2,4-dichlorophenoxyacetic acid (2,4-D), picloram and dicamba.

The term “endogenous” as used in the context of the present invention in combination with a protein or nucleic acid means that said protein or nucleic acid is still contained within the plant, i.e. is present in its natural environment. Often an endogenous gene will be present in its normal genetic context in the plant.

“Plant hormones”, “plant growth hormone”, “plant growth regulator” or “phytohormone” is a chemical that influences the growth and/or development of plant cells and tissues. Plant growth regulators comprise chemicals from the following five groups: auxins, cytokinins, gibberellins, abscisic acid (ABA) and ethylene. In addition to the five main groups, two other classes of chemical are often regarded as plant growth regulators: brassinosteroids and polyamines.

“Targeted mutagenesis” is mutagenesis that can be designed to alter a specific nucleotide or nucleic acid sequence, such as but not limited to, oligonucleotide-directed mutagenesis, mutagenesis using RNA-guided endonucleases (e.g. the CRISPR-technology), meganucleases, TALENs or Zinc finger technology.

The term “sequence of interest” includes, but is not limited to, any genetic sequence preferably present within a cell, such as, for example a gene, part of a gene, or a non-coding sequence within or adjacent to a gene. The sequence of interest may be present in a chromosome, an episome, an organellar genome such as mitochondrial or chloroplast genome or genetic material that can exist independently to the main body of genetic material such as an infecting viral genome, plasmids, episomes, transposons for example. A sequence of interest may be within the coding sequence of a gene, within transcribed non-coding sequence such as, for example, leader sequences, trailer sequence or introns. Said sequence of interest may be present in a double or a single strand nucleic acid molecule. The nucleic acid sequence is preferably present in a double-stranded nucleic acid molecule. The sequence of interest may be any sequence within a nucleic acid, e.g., a gene, gene complex, locus, pseudogene, regulatory region, highly repetitive region, polymorphic region, or portion thereof. The sequence of interest may also be a region comprising genetic or epigenetic variations indicative for a phenotype or disease. Preferably, the sequence of interest is a small or longer contiguous stretch of nucleotides (i.e. a polynucleotide) of duplex DNA, wherein said duplex DNA further comprises a sequence complementary to the sequence of interest in the complementary strand of said duplex DNA. The sequence of interest may be, or may be part of, a gene of interest, preferably an endogenous gene of interest.

### DETAILED DESCRIPTION

The inventors discovered that one or more cells and/or a tissue of a recalcitrant plant, optionally carrying a heritable mutation or transgene, can be straightforwardly regenerated when in contact with one or more cells and/or a tissue of a regenerative plant. The invention therefore pertains to regeneration and clonal and/or vegetative propagation of a recalcitrant plant. In addition, the invention concerns a method for producing a plant, preferably a recalcitrant plant, carrying a heritable mutation or a transgene, preferably at least in a clonally propagated tissue or plant part and/or in a germline progenitor cell for sexual reproduction. Hence, in an aspect, the invention provides for a method of increasing or inducing the regeneration capacity and/or efficiency of a cell of a recalcitrant plant, wherein said method comprises a step of contacting said cell with a cell of a regenerative plant.

The inventors discovered that shoots comprising or consisting of cells or tissue of the recalcitrant plant can be readily generated using such method, wherein “of the recalcitrant plant” is to be understood herein as being regenerated from the cell of the recalcitrant plant that is contacted with the cell of a regenerative plant in the method of the invention.

Therefore, in an aspect, the invention provides for a method of generating and selecting a shoot of a plant, wherein the method comprises the steps of:
(a) contacting a cell of a recalcitrant plant with a cell of a regenerative plant;
(b) allowing the contacted cells of step (a) to form one or more shoots; and
(c) selecting a shoot formed in step (b), wherein at least part of said shoot consists of cells of the recalcitrant plant.

Step (b) preferably comprises the formation of several shoots, i.e. more than one shoot. Therefore step (c) of the method of the invention may comprise the step of selecting a shoot from multiple shoots formed in step (b), wherein at least part of said selected shoot consists of cells of the recalcitrant plant.

Preferably, the recalcitrant plant or cell thereof is a plant or cell which fails to regenerate under normal growth conditions or that shows a poor regeneration efficiency. Optionally, the recalcitrant plant or cell thereof fails to regenerate or shows a poor regeneration efficiency under conditions known in the art to be optimal for regeneration, such as, but not limited to, conditions that allow for regeneration in the presence of externally supplied growth regulators. Although within species both recalcitrant and regenerative cultivars, varieties and/or accessions may exist, in general, pepper, soybean and sugar beet are non-limiting examples of recalcitrant plants, and cells thereof are non-limiting examples of recalcitrant plant cells. However, the method of the invention can be applied to all plants or plant cells that in some circumstance benefit from an increase in regeneration efficiency. Therefore, the recalcitrant plant or plant cell of step (a) of the method of the invention in particular is a plant or plant cell that shows less regeneration efficiency than the regenerative plant or plant cell of step (a) of the method of the invention. In a preferred embodiment, the recalcitrant plant or plant cell of step (a) of the method of the invention shows less regeneration efficiency than the regenerative plant or plant cell of step (a) of the method of the invention under conditions that are suitable, preferably optimal, for regeneration of the regenerative plant or plant cell. Such suitable and/or optimal conditions at least comprise suitable nutrient supply, optionally supplemented with hormones. Such conditions may further encompass a suitable and/or optimal temperature and/or light/dark regime. Preferably, such suitable and/or optimal conditions are applied in step (b) of the method of the invention. Preferably, the recalcitrant plant or plant cell of step (a) is a plant or plant cell that shows less regeneration efficiency and/or capacity as compared to the regenerative plant or plant cell of step (a) when exposed to similar conditions as applied in step (b) of the method of the invention with the exception that said recalcitrant plant or plant cell is not in contact with said regenerative plant or plant cell. Put differently, the recalcitrant plant or plant cell of step (a), is a plant or plant cell that shows less regeneration efficiency and/or capacity as compared to the regenerative plant or plant cell of step (a) when the recalcitrant and regenerative cell are exposed to similar conditions as applied in step (b) of the method of the invention, preferably in the absence of a contacting step (a) as defined herein. These conditions are preferably conditions suitable for the regenerative plant, or cell thereof, to regenerate. The skilled person is aware of conditions suitable for regenerative plant cells to regenerate. Such conditions may be conditions under which the recalcitrant plant cell normally (i.e. when not in contact with a regenerative cell) does not show, are hardly shows, regeneration.

Preferably, step (b) of the method of the invention is performed under conditions suitable for the regenerative plant or cell thereof to regenerate. Optionally, step (b) of the method of the invention may be performed under conditions under which the cell of the recalcitrant plant, unlike the cell of the regenerative plant, shows no or hardly any regeneration when not in contact with said regenerative plant cell.

Preferably, the recalcitrant plant cell and the regenerative plant cell of step (a) of the method of the invention are somatic cells. Optionally, the recalcitrant plant or plant cell is modified to comprise a positive selection marker. Typical non-limiting examples of plants known in the art to be recalcitrant are pepper (Capsicum annuum), sugarbeet (Beta vulgaris, more in particular Beta vulgaris subsp. vulgaris), soybean (Gycine max), sunflower (Helianthus annuus), cotton (Gossipium hirsutum), hemp or cannabis (Cannabis sativa), strawberry (Fragaria×ananassa), hops (Humulus lupulus), melon (Cucumis melo) and cucumber (Cucumis sativus). The recalcitrant plant may be a plant which shows no or hardly any regeneration, but may also be a plant for which regeneration can be (further) improved. Therefore, and as exemplified herein, the recalcitrant plant may be, but is not limited to, a Capsicum annuum, a Solanum tuberosum or Taraxacum brevicorniculatum. Likewise, the cell of the recalcitrant plant may be, but is not limited to, a cell of Capsicum annuum, a Solanum tuberosum or Taraxacum brevicorniculatum.

Preferably, the regenerative plant (or cell thereof) of step (a) of the method of the invention is capable of regeneration under normal growth conditions, preferably conditions that allow for the regeneration of the plant (or cell thereof) in the absence of externally supplied growth regulators such as auxins and/or cytokinines. Preferably under such conditions, the regenerative plant may form de novo shoots on a multicellular tissue. The regeneration is preferably at least one of organogenesis and somatic embryogenesis. Preferably, the regenerative plant is capable of regenerating shoots after decapitation in vivo, i.e. removal of all preformed shoot apical meristems. Although within species both recalcitrant and regenerative cultivars, varieties and/or accessions may exist, in general, seedling hypocotyls of tomato and tobacco, Capsicum baccatum, Solanum melongena, Solanum tuberosum are known in the art as examples of regenerative explants.

The regenerative plant or plant cell may be a naturally occurring regenerative plant or plant cell, i.e. a plant or plant cell that has a natural ability to regenerate. Alternatively, the plant or plant cell may be genetically modified to increase the regeneration potential. Examples of such plants or plant cells include, but are not limited to, plants and plant cells disclosed in WO2019/211296 and WO2019/193143, which are incorporated herein by reference. As a non-limiting example, the regenerative plant or plant cell can be a plant or plant cell modified to have induced or increased expression of a histidine kinase selected from the group consisting of CHK4, CHK2 and CHK3, preferably as described in WO2019/193143. In addition or alternatively, the regenerative plant or plant cell is a plant or plant cell modified to have, preferably transiently, induced or increased expression of transcription factors associated with regeneration, preferably

at least one of a WUSCHEL related homeobox protein (preferably WOX5, optionally AtWox5 of SEQ ID NO: 16), a PLETHORA protein (preferably PLT1, optionally AtPLT1 of SEQ ID NO: 17) and WOUND INDUCED DEDIFFERENTIATION 1 protein (WIND1, optionally AtWIND1 of SEQ ID NO: 18), preferably both WOX5 and PLT1, even more preferably WOX5, PLT1 and WIND1, as described in WO2019/211296. Preferably, said transcription factors are under the control of an inducible promoter and regeneration is induced by exposing the cells to the agent resulting in the induction of said inducible promoter. Optionally, the regenerative plant or plant cell is a plant or plant cell that is transfected by the SHOOT REGENERATION-2 vector or the SHOOT REGENERATION vector as described in WO2019/211296. Said vector may be introduced by transient or stable transfection and regeneration may be induced by exposing the contacted cells to at least one of dexamethasone and estradiol, preferably to both dexamethasone and estradiol, as the indicated transcription factors associated with regeneration are under the control of promoters that are inducible through administration of these compounds (referred in this respect is to WO2019/211296). In addition or alternatively, the regenerative plant may be a plant that has a mutation in an endogenous gene resulting in increased regeneration capacity and/or efficiency. Non-limiting examples are known in the art, e.g. the ATHB15 mutant described in Duclerq et al. (Plant biology, 2011, 13, p317-324), the KCS1 mutant as described in Shang et al. (PNAS 2016, 113, 5101-5106), ARR mutants as described in Buechel et al. (European Journal of Cell Biology 2010, 89:279-284) and ATRXR2 mutant as described in Lee et al. (2021) Cell Reports 37, 1-13.

Optionally, the regenerative plant or plant cell is modified to comprise a negative selection marker. A regenerative plant or plant cell may be, but is not limited to, a Capsicum baccatum, Solanum lycopersicum and Cichorium intybus. In particular, the regenerative plant or plant cell of step (a) of the method of the invention is a plant or plant cell that shows a higher regeneration efficiency as compared to the recalcitrant plant or plant cell of step (a) of the method of the invention. Preferably the regenerative plant or plant cell of step (a) of the method of the invention is a plant that shows a higher regeneration efficiency as compared to the recalcitrant plant or plant cell of step (a) of the method of the invention under conditions that are suitable, preferably optimal, for regeneration of the regenerative plant or plant cell. Such suitable and/or optimal conditions at least comprise suitable nutrient supply, optionally supplemented with hormones. Such conditions may further encompass a suitable and/or optimal temperature and/or light/dark regime.

Preferably, the recalcitrant plant or plant cell of step (a) of the method of the invention has a different genotype than the regenerative plant or plant cell of step (a) of the method of the invention. The recalcitrant plant or plant cell and the regenerative plant or plant cell of step (a) of the method of the invention can be of a different genus or of the same genus. Preferably, the recalcitrant plant or plant cell and the regenerative plant or plant cell of step (a) of the method of the invention are of the same genus. Optionally, the recalcitrant plant cell and the regenerative plant cell of step (a) of the method of the invention are of a different species. Preferably, the regenerative plant or plant cell of step (a) of the method of the invention is a plant or plant cell that is capable of exchanging genetic material through traditional breeding methods with the recalcitrant plant or plant cell of step (a) of the method of the invention. Preferably, a cell of the recalcitrant plant can hybridize with a cell of a regenerative plant.

As a non-limiting example, the recalcitrant plant cell and the regenerative plant cell can both be of the genus Solanum or of the genus Capsicum. For example, the recalcitrant plant or cell thereof can be a Capsicum annuum and the regenerative plant or cell thereof can be a Capsicum baccatum. Similarly, the recalcitrant plant or cell thereof can be a Solanum tuberosum and the regenerative plant or cell thereof can be a Solanum lycopersicum. Further, the recalcitrant plant or cell thereof can be a Taraxacum brevicorniculatum and the regenerative plant or cell thereof can be a Cichorium intybus.

In a preferred embodiment, the recalcitrant plant or plant cell of step (a) of the method of the invention is a plant that shows a lower regeneration capacity and/or efficiency as compared to the regenerative plant or plant cell of step (a) of the method of the invention, under the same or similar conditions that allow for regeneration, but without making contact to one another. Said conditions are preferably normal growth conditions suitable for both the regenerative and recalcitrant plant or plant cells thereof. More preferably, said conditions are conditions that allow for regeneration, even more preferably, said conditions are conditions that allow for regeneration in the absence of externally supplied (e.g. the addition of chemicals through human interference) growth regulators. In a preferred embodiment, said conditions are at least the minimal required conditions for regeneration of the regenerative plant or plant cell. Optionally, said conditions are at least the suitable conditions and optionally the optimal conditions for regeneration of the regenerative plant or plant cell.

In an aspect, the invention pertains to a method of generating and selecting a shoot of a plant, wherein the selected shoot comprises germline progenitor cells of a recalcitrant plant, i.e. germline progenitor cells regenerated from the recalcitrant plant cell of step (a) of the method of the invention. Hence, optionally, the method of the invention comprises the steps of:
(a) contacting a cell of the recalcitrant plant with a cell of a regenerative plant;
(b) allowing the contacted cells of step (a) to form one or more shoots; and
(c) selecting a shoot formed in step (b), wherein said shoot comprises germline progenitor cells of the recalcitrant plant.

In addition or alternatively, the invention pertains to a method of generating and selecting a shoot of a plant, wherein the shoot comprises cells giving rise to clonally propagated tissue and/or a plant part of the recalcitrant plant, i.e. clonally propagated tissue and/or plant part regenerated from the recalcitrant plant cell of step (a) of the method of the invention. Hence, optionally, the method of the invention comprises the steps of:
(a) contacting a cell of the recalcitrant plant with a cell of a regenerative plant;
(b) allowing the contacted cells of step (a) to form one or more shoots; and
(c) selecting a shoot formed in step (b), wherein said shoot comprises cells giving rise to clonally propagated tissue and/or a plant part of the recalcitrant plant.

Optionally, the contacted cell of the recalcitrant plant forms callus in step (b), and the shoots are grown from the callus. Therefore in an embodiment, the method of the invention is a method of generating, and optionally selecting, a shoot of a plant comprising germline progenitor cells of a recalcitrant plant, wherein the method comprises the steps of:
(a) contacting a cell of the recalcitrant plant with a cell of a regenerative plant;
(b) allowing the contacted cell of the recalcitrant plant of step (a) to form callus and allowing shoots to grow from the callus; and
(c) selecting a shoot obtained in step (b) comprising germline progenitor cells of the recalcitrant plant.

Therefore, preferably the method of the invention comprises a step (c) of selecting a shoot, wherein at least part of said shoot consists of cells of the recalcitrant plant (i.e. being regenerated from the recalcitrant plant cell of step (a)), and wherein said part is at least one of:
i) a tissue comprising germline progenitor cells; and
ii) a tissue comprising cells giving rise to a clonally propagated tissue and/or a clonally propagated plant part.

Clonally propagated tissue and/or plant part is understood herein as a tissue and/or plant part that can be used for clonal propagating into offspring, i.e. a plant of a subsequent generation. Such tissue and/or plant part may be, but is not limited to, a tuber, bulb, corm, cormel, sucker, slip, crown, bulbil, rhizome, apical portion of stem, shoot or root cutting, basal knob or truncheon, stolon, tuberous stem cutting or eye, (clonally propagated) seed, and the like. The genotype of the (cells giving rise to) clonally propagated plant part or tissue therefore determines the genotype of offspring of clonally propagated plants and any genomic modification made in (cells giving rise to) this tissue or part may be carried on to the subsequent generation(s). Hence a transgene or mutation made in (cells giving rise to) a clonally propagated plant part is a heritable transgene or mutation.

Germline progenitor cells are understood herein as those cells, or their clonal descendants, that will ultimately differentiate into gametes. The genotype of the germline progenitor cell therefore determines the genotype of the gametes and any genomic modification made in a germline progenitor cell will be carried on to the subsequent generation(s). Hence a transgene or mutation introduced in a germline progenitor cell is heritable, i.e. an heritable transgene or a heritable mutation. The L2-shoot meristem layer may determine the genotype of the gametes (see e.g. Filippis et al. Using a periclinal chimera to unravel layer-specific gene expression in plants, The Plant Journal, 2013, 75:1039-1049). Preferably, in step (c) the part of the selected shoot that consists of cells of the recalcitrant plant is a tissue comprising germline progenitor cells. Hence preferably, the germline progenitor cells of the method of the invention are derived from the L2-shoot meristem layer of the recalcitrant plant (i.e. an L2-meristem layer regenerated from the recalcitrant plant cell of step (a) of the method of the invention). The shoot selected in step (c) of the method of the invention may further comprise at least one of an L1 and L3-shoot meristem layer of the recalcitrant plant. Optionally, said shoot comprises the L1, L2 and L3-shoot meristem layer of the recalcitrant plant. Alternatively, the shoot selected in step (c) of the method of the invention may comprise an L2-shoot meristem layer regenerated from the recalcitrant plant cell of step (a) and at least one of the L1- and L3-shoot meristem layer regenerated from the regenerative plant cell of step (a). The shoots, or at least one shoot, grown in step (b) and selected in step (c) of the method of the invention may be adventitious shoots, or at least one adventitious shoot.

In step (a) of the method of the invention, a cell of the recalcitrant plant is brought into contact with a cell of a regenerative plant. Optionally, in step (a) of the method of the invention, one or more cells of the recalcitrant plant are brought into contact with one or more cells of a regenerative plant. The step (a) of contacting can be performed using any conventional method known to the skilled person. The contacting step preferably requires that a cell of a recalcitrant plant is brought in in physical contact with a cell of a regenerative plant. Physical contact of cells is to be understood herein as the stable association or aggregation of cells through cell-cell contact, optionally via a reagent or linker aggregating the cells. Preferably, plasmodesmata are formed between cells having physical contact. Preferably, the cell-cell contact is such that a common cell wall is formed. Hence, preferably the contact between the cell of a recalcitrant plant and the cell of a regenerative plant results in e.g. the sharing of a cell wall and/or plasmodesmata.

As non-limiting example, this contacting step may include any in-vitro technique known in the art, e.g. conventional techniques for producing a periclinal chimera: These techniques may include at least one of the following:

(1) Co-culturing of cells, wherein adjoined stem slices from a regenerative plant and a recalcitrant plant are cultured together into callus, and adventitious shoots are regenerated from these calli, preferably on hormone-supplemented in vitro growth media.

(2) Mixed callus cultures, wherein cell-suspensions of a regenerative plant and a recalcitrant plant are mixed, the mixtures are grown into callus, and adventitious shoots are regenerated from these calli, preferably on hormone-supplemented in vitro growth media.

(3) Co-culture of protoplasts, wherein protoplast suspensions of a regenerative plant and a recalcitrant plant are embedded in optionally agarose and grown at very high cell densities, upon which shoots are regenerated, preferably on hormone-supplemented in vitro growth media.

(4) In vitro graft culture, wherein a regenerative plant and a recalcitrant plant are grafted along their hypocotyls or internodes, preferably under sterile conditions. One of the plants is “the scion” and the other one “the rootstock”. Cross sections of the graft junction are cultured to induce adventitious calli and shoots. Such techniques fall under the common denominator of tissue culture, and consist of a multitude of distinct protocols that may be specific for individual plant lines or species. The skilled person will know how to bring cells of two different plants together in tissue culture, to regenerate shoots. For an elaborate review on plant chimeras, see “Plant Chimeras” by Richard A. E. Tilney-Bassett (Cambridge University Press, 1991). Optionally, any one of the above techniques is practiced, optionally as further specified herein, in the method of the invention, thereby generating at least one (adventitious) shoot comprising or consisting of tissue regenerated from the recalcitrant plant cell, preferably said tissue comprises germline progenitor cells, and/or gives rise to a clonally propagated tissue and/or a clonally propagated plant part.

Optionally, the cell of the recalcitrant plant and the cell of the regenerative plant in step (a) of the method of the invention are comprised in a tissue and/or plant part. The contacting step (a) of the method of the invention can be performed by grafting of a recalcitrant plant and a regenerative plant, one as rootstock and the other one as scion. Preferably, the rootstock is a regenerative plant and the scion is a recalcitrant plant. Hence preferably, the contacting of step (a) is performed by grafting a scion of a recalcitrant plant on the stock of the regenerative plant. Alternatively, the rootstock may be the recalcitrant plant and the scion may be the regenerative plant, and the contacting of step (a) is performed by grafting a scion of a regenerative plant on the rootstock of the recalcitrant plant. Grafting in general comprises the step allowing a scion and a rootstock to join together in such a way that the vascular tissues grow together to form a graft junction.

Preferably, the shoot removed from the rootstock plant comprises the apical bud, thereby rendering a “decapitated” plant. Shoot decapitation is preferably in the hypocotyl or epicotyl, or internode. Preferably, the cotyledonary node is grafted onto a decapitated hypocotyl stock. At the graft junction a thin strip of callus may be formed. The grafting may be a grafting of a scion onto a rootstock, also indicated herein as stock-scion grafting. However, the skilled person understands that variations are possible and these are included in the method of the invention. As a non-limiting example, a section or “slice” of a regenerative plant may be inter-grafted in between a rootstock and a scion of a recalcitrant plant. Preferably, young plant material is used for grafting, wherein said young plant material is seedling material of between 1-4, or between 1-3 weeks after sowing, preferably using material of about 2 weeks after sowing. Preferably, the young plant material used for grafting is seedling material of between 0.1 and 1 mm, between 0.1 and 0.75 mm, between 0.1 and 0.5 mm, or between 0.1 and 0.25 mm. Preferably, seedling material is used just after development of the first true leaves. Preferably, in said grafting process, (tiny) steel pins, preferably sterile steel pins, are used for alignment and fixation of said seedling material. Said steel pins can be inserted in the centre of the stock and scion as exemplified herein (e.g. see FIG. 1). In addition or alternatively, ties, tapes, bands, and/or clamps may be used around the two grafting partners to hold them together, and optionally an adhesive (glue, wax or paste) may be used at edges of the graft junction for fixation.

In a graft junction, the vascular tissue s of the rootstock and scion are connected with each other, allowing nutrients and water to transfer from the rootstock to the scion. It is to be understood that the recalcitrant plant and the regenerative plant within this embodiment of the invention, i.e. making use of grafting in the contacting step (a) of the method of the invention, are plants that are naturally capable of forming graft junctions. Preferably, the recalcitrant plant and the regenerative plant are both dicotyledonous plants. Optionally, the recalcitrant plant and the regenerative plant are both monocotyledonous plants. Optionally, grafting is performed using young plant material, e.g. young plant material obtained from in vitro micropropagation, young seedling material or seeds. Such methods for grafting monocotyledoneous plants are provided e.g. in WO2020/099878 and WO2020/099879, which are incorporated herein by reference.

Graft unions, after healing, can be subsequently cut or “wounded” resulting in the production of callus, and adventitious shoots. Among these adventitious shoots, shoots comprising or consisting of recalcitrant plant cells, e.g. comprising germline progenitor cells of the recalcitrant plant and/or clonally propagated plant tissue and/or plant parts of the recalcitrant plant, can appear spontaneously.

The cut is preferably made at the intersection between the recalcitrant and the regenerative plant of the graft union. Preferably, the cut is such that a thin layer of scion cells is left on top. Preferably, these scion cells are cells from a recalcitrant plant. Preferably, the cut is just at the graft junction healing. Hence, preferably step (b) may comprise a step of generating a wound at or near the graft junction and allowing callus to be formed at the wounded graft junction.

The wound may be a complete cut, e.g. a transverse cut, separating the graft into two plant parts. Optionally, the wound does not completely separate the graft union into two plant parts, but is sufficient to initiate and/or stimulate the production of callus. Optionally, wounding is performed by taking a slice of tissue from the graft junction, and subsequently culturing the slice to allow callus to form. Preferably, a shoot is grown from said callus, wherein said shoot may comprise or consist of tissue regenerated from the recalcitrant plant cell. In other words, preferably a shoot is grown from said callus, wherein at least part of said shoot consists of cells of the recalcitrant plant. This particular method is practiced under ambient conditions, in a growth room or greenhouse.

Therefore, optionally, the contacting of step (a) is performed by grafting a scion of a recalcitrant plant on the stock of the regenerative plant and allowing the graft junction to heal. Optionally, said method further comprises in step (b) the step generating a wound just at or near the graft junction, allowing callus to be formed at the (wounded) graft junction and allowing a shoot to grow from said callus, and wherein at least part of said shoot consists of cells of the recalcitrant plant.

In a preferred embodiment, the method of the invention is a method for generating and selecting a shoot, comprises the steps of:
(a) contacting a cell of the recalcitrant plant with a cell of a regenerative plant, by grafting a scion of one of the recalcitrant plant and regenerative plant on the rootstock of the other of the recalcitrant plant and regenerative plant and allow the formation of a graft junction between the scion and rootstock in order to obtain a graft union;
(b) allowing the contacted cells of the recalcitrant plant of step (a) to form callus by generating a wound at or near the graft junction, and allowing one or more shoots to grow from the callus; and
(c) selecting a shoot grown in step (b) wherein at least part of said shoot consists of cells of the recalcitrant plant.

Optionally, one or more cells of the recalcitrant plant within the selected shoot in step (c) are germline progenitor cells. Optionally, the cells of the recalcitrant plant within the selected shoot in step (c) give rise to clonally propagated tissue and/or plant parts.

Preferably, in step (a) the regenerative plant and recalcitrant plant are seedlings or young vegetative (optionally in vitro) cloned plants, preferably as defined herein, and grafting can be facilitated by inserting a steel pin in the centre of said scion and rootstock for alignment and fixation.

As indicated above, alternative methods are available for contacting cells of different plants.

In an alternative embodiment, the contacting step (a) of the method of the invention can be performed by contacting one or more isolated cells of the recalcitrant plant with one or more isolated cells of the regenerative plant. Therefore, the cell of the recalcitrant plant and the cell of the regenerative plant of step (a) can be isolated cells, preferably protoplasts. Such isolated cells may be single cells. Preferably, the contacting step (a) is performed by contacting a protoplast of a recalcitrant plant with a protoplast of a regenerative plant. Preferably, the isolated cells in step (a) of the method of the invention are treated with a compound promoting aggregation of the cell membranes of the cells.

Preferably, within this process, the one or more isolated cells of both the recalcitrant and the regenerative plants are protoplasts. In a preferred embodiment, protoplasts of recalcitrant and regenerative plants are contacted using a method known in the art that promote adhesion of the membranes of these protoplasts, such as, but not limited to aggregation mediated by at least one of polyethylene glycol (PEG), Yariv antigen or reagent (1,3,5-tris (4-β-D-glycopyranosyloxyphenylazo)-2,4,6-trihydroxy-benzene), biotin-streptavidin and antibodies (e.g. see Yariv et al. (1962) Biochem. J. 85, 383-388; Hartmann et al. Planta (Berl.) 1973, 112; 45-56; Larkin, J. Cell Sci. 1978, 30; 283-282; Kesteren and Tempelaar, Plant Science 1993, 93; 131-141). Such method may comprise a treatment that promotes protoplasts to come into close proximity to one another, for instance by treatment with PEG, preferably using a solution comprising about 15% PEG, wherein said PEG preferably is PEG 3350 MW. Preferably, said PEG solution is a PEG solution as exemplified in the Examples herein below. Alternatively or in addition, such method may comprise a treatment to neutralize the normal surface charge so that agglutinated protoplasts can come in intimate contact, such as the treatment with a high Ca²⁺ ion concentration and a high pH, preferably a concentration of about 50 mM Ca²⁺ ions at a pH of at least 10, preferably at pH 10.5. Preferably, said neutralizing solution is a neutralizing solution as exemplified in the Examples herein below (Example 2). In a preferred embodiment, step (a) of the method of the invention comprises the following (sub-)steps:
(a1) isolation of protoplasts of a regenerative plant and protoplasts of a recalcitrant plant;
(a2) mixing the isolated protoplasts of the recalcitrant plant with the isolated protoplasts of the regenerative plant;
(a3) treating the mixture with PEG; and
(a4) treating the mixture with a neutralizing solution.

Optionally, prior to step (a2) and after step (a1), the protoplasts of the regenerative plant are treated with an agent inhibiting mitosis.

In addition or alternatively, such method may comprise a step of promoting adhesion or agglutination of the protoplasts by linking cell membranes of plant cells and/or protoplasts together. In other words, preferably a linking reagent that links the cell membranes of plant cells together can be used in step a) of the method of the invention.

Preferably, the linking reagent binds, preferably selectively binds, galactans and/or arabinogalactan proteins (AGPs) present on the plant cells, thereby linking the plant cells together.

A preferred linking reagent is Yariv. Yariv (1,3,5-tri(p-glycosyloxyphenylazo)-2,4,6-trihydroxybenzene) is a compound capable of linking protoplasts. Preferably, in the method of the invention, Yariv is used that comprises glycosyl groups that are selected from the group consisting of glucose, galactose or mannose, maltose, xylose, lactose and cellulose. Preferably Yariv comprises three β-D glycosyl groups. Therefore, in a further preferred embodiment, step (a) of the method of the invention comprises the following (sub-)steps:
(a-i) isolation of protoplasts of a regenerative plant and protoplasts of a recalcitrant plant;
(a-ii) mixing the protoplasts of the regenerative plant with the protoplasts of a recalcitrant plant;
(a-iii) treating the mixture with a protoplast-linking reagent; and
(a-iv) recovering the linked protoplasts.

In step (a-iv), preferably the (linked) protoplasts are washed extensively, preferably with a protoplast medium such as, but not limited to 9M (9% (w/v) mannitol, 140 mg/L CaCl₂·H₂O, 580 mg/L MES at pH 5.8). Preferably, subsequently, the contacted and/or linked protoplasts of step (a) are embedded in a hydrogel, preferably alginate polymer, for culturing in step (b) of the method of the invention.

The invention also provides for the use of an agent linking plant cell membranes for regeneration or a recalcitrant plant cell as defined herein. Preferably, said linking agent being β-D-galactosyl Yariv.

The protoplasts of the regenerative plant and the protoplasts of a recalcitrant plant are preferably admixed in a ratio of about 1:1, 1:2, 1:3, 1:4, 1:5, etc prior to contacting and/or linking, depending on the cell types and/or species. An abundance of recalcitrant plant cells over regenerative plant cells may be preferred to prevent that the regenerative plant cells may outcompete the recalcitrant plants cells. Preferably, the contacting in step (a) is performed by forcing together and thereby further increasing the contact between the mixed protoplasts (preferably about 10 to about 100 protoplasts), preferably after or during treating the protoplast mixture with a linking agent as indicated herein (i.e. during or after step (a-iii)). This can be achieved using a mesh as exemplified herein such that multiple protoplasts are physically contacted to one another in the holes of the mesh. In a preferred method, multiple protoplasts are contacted to one another during treatment of the protoplasts with a linking agent in step (a) of the method of the invention. Linking, preferably with Yariv reagent, is preferably performed about 15 to about 30 minutes.

Step (b) is performed under conditions that allow for regeneration of the regenerative plant or plant cell, preferably conditions suitable for shoot formation. These conditions are understood herein as at least being the minimal requirements of the regenerative plant or plant cell to regenerate, which in general at least include normal growth conditions of said plant or plant cell. Preferably, step (b) is performed under conditions suitable for the regenerative plant or regenerative plant cell of step (a) to regenerate. Preferably, step (b) comprises the formation of callus prior to shoot formation. Therefore, step (b) may comprise the sub-steps of (b1) allowing the contacted cells of at least the recalcitrant plant to form callus; and (b2) allowing a shoot to grow from said callus, wherein optionally the culturing conditions of (b1) and (b2) are different. More in particular, step (b1) may be performed under conditions suitable for the cell of at least the regenerative plant to form callus; and step (b2) may be performed under conditions suitable for the regenerative plant callus to form shoots. The skilled person is aware of conditions suitable for callus and/or shoot regeneration for regenerative plants. Preferably, in step (b) in addition to the recalcitrant plant cell, also the regenerative cell regenerates to form shoot. Optionally, in addition to the recalcitrant plant cell, also the regenerative cell in step (b1) regenerates to form callus, and optionally, said callus also regenerates in step (b2) to form shoots. Therefore, optionally, in step (b), (b1) and/or (b2) the regenerative cell and the recalcitrant cell, co-regenerate.

Callus may be formed during the regeneration process of step (b) by (shoot) organogenesis or somatic embryogenesis. The amount of formed callus may be dependent on e.g. the plant species used in the method of the invention and/or the used conditions that allow for shoot formation. Said callus formation, e.g. in vitro and/or after grafting and wounding, may occur spontaneously, i.e. in the absence of one or more externally supplied plant hormones. Similarly, the formation of shoots in step (b), e.g. after optional callus formation, may occur spontaneously, thus in the absence of one or more externally supplied plant hormones. Alternatively, said formation of callus may be induced and/or augmented in the presence of one or more plant hormones. Alternatively or in addition, the formation of shoots in step (b) may be induced and/or augmented in the presence of one or more plant hormones.

In a particular embodiment, formation of callus is minimal between the contacting of the cells in step (a) and the formation of a shoot in step (b) of the method of the invention. This is in particular preferred in order to avoid the cells of regenerative plant to outcompete the cells of the recalcitrant plant. A minimal callus stage may therefore increase the chance of growing a shoot that comprises or consists of cells of the recalcitrant plant.

For the induction of shoot regeneration in plant tissues, a combination of one or more cytokinins and one or more auxins may be employed.

The cytokinin that may be used in the method of the invention can be an adenine-type cytokinin or a phenylurea-type cytokinin. Similarly, the cytokinin can be a naturally produced phytohormone or can be a synthesized compound. The adenine-type cytokinin can be a phytohormone that is synthesized in at least one of roots, seeds and fruits. In addition, cambium and other actively dividing tissues can also synthesize cytokinins. A non-limiting example of a naturally occurring adenine-type cytokinin is Zeatin as well as its metabolic precursor 2iP. Non-limiting examples of synthetic adenine-type cytokinins are kinetin and 6-benzylaminopurine (BAP). Substituted urea compounds, such as thidiazuron and CPPU do not occur in plants but can act as cytokinins in tissue culture. The adenine-type cytokinin can be selected from the group consisting of kinetin, zeatin, trans-zeatin, cis-zeatin, dihydrozeatin, 6-benzylaminopurine and 2iP, and combinations thereof. The phenylurea-type cytokinin can be diphenylurea or thidiazuron. It is known in the art that the type of added cytokinin is dependent on the type of plant cell and the skilled person can straightforwardly select the suitable cytokinin(s), if needed.

Alternatively or in addition, the plant hormone may be an auxin. The auxin can be an endogenously synthesized auxin. The endogenously synthesized auxin can be selected from the group consisting of indole-3-acetic acid (IAA), 4-chloroindole-3-acetic acid, phenylacetic acid, indole-3-butyric acid and indole-3-propionic acid. The auxin can be a synthetic auxin, e.g. an auxin analog. The synthetic auxin can be at least one of 1-naphthaleneacetic acid, 2,4-dichlorophenoxyacetic acid (2,4-D), α-Naphthalene acetic acid (α-NAA), 2-Methoxy-3,6-dichlorobenzoic acid (dicamba), 4-Amino-3,5,6-trichloropicolinic acid (tordon or picloram), 1-naphthaleneacetic acid (NAA), indole-3-butyric acid (IBA) and 2,4,5-trichlorophenoxyacetic acid (2,4,5-T). The auxin can be 1-naphthaleneacetic acid (NAA).

For shoot organogenesis by a combination of cytokinin and auxin, preferably the cytokinin to auxin ratio should preferably is >1 (Dodds, J H and Roberts, L W (1985) Experiments in plant tissue culture. Cambridge University Press, Cambridge, UK).

Optionally, in step (b) first callus formation is stimulated (step b1) and subsequently shoot formation is stimulated (step b2). Step (b1) may be performed using conditions allowing for callus formation of the regenerative plant. Optionally, step (b1) is performed using minimal conditions allowing for callus formation of the regenerative plant. In a preferred embodiment, step (b1) is performed using optimal conditions for callus formation of the regenerative plant. Step (b2) may be performed using conditions allowing for shoot formation of the regenerative plant. Optionally, step (b2) is performed using minimal conditions allowing for shoot formation of the regenerative plant. In a preferred embodiment, step (b2) is performed using optimal conditions for shoot formation of the regenerative plant.

The method of the invention may further comprise a step (d) of growing a plant from the shoot selected in step (c).

Optionally, in step (c) of the method of the invention, a shoot is selected that comprises germline progenitor cells of the recalcitrant plant. Such shoot can give rise to a plant comprising germline progenitor cells and/or germline cells (e.g. gametes, egg cell, sperm cell) that are derived from the recalcitrant plant cell used in step (a). Germline cells may form gametes for sexual reproduction. Such plant can be subsequently used to produce seed, wherein said seed comprises an embryo, and wherein at least part of the genotype of the embryo is derived from the recalcitrant plant cell of step (a) of the method of the invention, optionally the seed is obtained by selfing or backcrossing.

Optionally, in step (c) of the method of the invention, a shoot is selected that comprises cells that may give rise to clonally propagated tissue or plant parts of the recalcitrant plant, i.e. may give rise to clonally propagated tissue and/or plant part regenerated from the recalcitrant plant cell of step (a) of the method of the invention. Such shoot can give rise to a plant comprising plant parts derived from the recalcitrant plant cell that can be used for clonal propagation. Such plant part has the same or substantially the same genotype as the recalcitrant plant cell of step (a) of the method of the invention.

The selected shoot may be substantially free of cells of the regenerative plant. Such shoot may consist of cells of the recalcitrant plant (i.e. of cells regenerated from the recalcitrant plant cell) and can be used to produce a recalcitrant plant by (vegetative) propagation of said shoot, i.e. by growing a whole plant from said shoot. The step of selecting the shoot can be performed using any conventional method known to the skilled person.

The selection may comprise a step of determining a phenotypic characteristic and/or a molecular marker that is present in the cells of the recalcitrant plant and/or present in a shoot meristem layer of the recalcitrant plant, but absent in the cells of the regenerative plant and/or absent in a shoot meristem layer of the regenerative plant. Alternatively or in addition, the selection may comprise a step of determining a phenotypic characteristic and/or a molecular marker that is absent in the cells of the recalcitrant plant and/or absent in a shoot meristem layer of the recalcitrant plant, but is present in the cells of the regenerative plant and/or present in a shoot meristem layer of a regenerative plant. Preferably, the selection may comprise a step of determining a phenotypic characteristic and/or a molecular marker that is present in the germline progenitor cells and/or the clonally propagated plant part of the recalcitrant plant, but not present in the germline progenitor cells and/or the clonally propagated tissue and/or plant part of the regenerative plant. Alternatively or in addition, the selection may comprise a step of determining a phenotypic characteristic and/or a molecular marker that is absent in the germline progenitor cells and/or the clonally propagated tissue and/or plant part of the recalcitrant plant, but is present in the germline progenitor cells and/or the clonally propagated tissue and/or plant part of the regenerative plant. The molecular marker is preferably a genomic sequence, that is present either in the recalcitrant plant or in the regenerative plant.

Alternatively or in addition, step (c) and/or (d) may comprise a step of bringing the (regenerated) shoot into contact with a compound that is toxic for (plant) cells that express a negative selection marker. In this embodiment, a negative selection marker is expressed in cells of the regenerative plant, preferably a negative selection marker is expressed in at least the germline progenitor cells and/or the clonally propagated tissue and/or plant part of the regenerative plant. Optionally, a toxic selection marker is encoded by (the genome of) the regenerative cell, optionally under the control of an inducible promoter. By exposure of such cells to a substance activating the inducible promoter, the toxic selection marker is expressed and preferably the regenerative cells die. Optionally, a precursor of a toxic selection marker is encoded by the (genome of) in the regenerative cell. By exposure of such cells to a substance that activates the conversion of the precursor to a toxic component, preferably thereby killing the regenerative cells.

Alternatively or in addition, step (c) and/or (d) may comprise a step of bringing the (regenerated) shoot into contact with a compound that is toxic for (plant) cells, but can be converted into a non-toxic compound by the expression of a positive selection marker. In this embodiment, a positive selection marker is expressed in a shoot meristem layer of the recalcitrant plant, preferably a positive selection marker is expressed in at least the germline progenitor cells and/or the clonally propagated tissue and/or plant part of the recalcitrant plant.

In a preferred embodiment, at least one or more germline progenitor cells and/or the clonally propagated tissue and/or plant parts of the generated shoot selected in step (c) of the method of the invention comprise a transgene or a mutation in a sequence of interest. Preferably, at least one of the L1-, L2- and/or L3-shoot meristem layer of the generated shoot comprises a transgene or mutation in a sequence of interest. Preferably, at least the L2-shoot meristem layer of the generated shoot comprises a transgene or mutation in a sequence of interest.

The transgene or mutation may be present in a cell of the recalcitrant plant of step (a) of the method of the invention and optionally in a cell of the regenerative plant of step (a) of the method of the invention. Preferably, the transgene or mutation is present in a cell of the recalcitrant plant of step (a) of the method of the invention. Preferably, the transgene or mutation is present at least in a germline progenitor cell and/or the clonally propagated tissue and/or plant part of the recalcitrant plant of the generated shoot selected in step (c) of the method of the invention. Optionally, the transgene or mutation is present in all or substantially all cells of the generated shoot selected in step (c), wherein said cells are cells regenerated from the recalcitrant plant cell of step (a) of the method of the invention. Preferably, at least the L2-shoot meristem layer of the generated shoot comprises a transgene or mutation in a sequence of interest and wherein at least the L2-shoot meristem layer is of the recalcitrant plant or, in other words, is regenerated from the recalcitrant plant cell provided in step (a). Subsequent seed produced from such shoot may comprise said transgene or mutation, preferably within the embryo of said seed.

Therefore preferably the method of the invention comprises the step of introducing transgene or a mutation in a sequence of interest in a cell of the recalcitrant plant of step (a). Alternatively or in addition, the method of the invention (further) comprises the step of introducing a transgene or mutation in a sequence of interest in a cell originating from the recalcitrant plant of step (a) and present in the shoot formed in step (b), optionally in the callus formed in step (b).

The transgene or mutation may be introduced into the cell (optionally in one or more cells) of the recalcitrant plant before the cell or cells are contacted with the cell (optionally the one or more cells) of a regenerative plant in step (a). As a non-limiting example the transgene or mutation can be introduced in one or more cells of the recalcitrant plant, followed by co-culturing these cells with one or more cells of the regenerative plant. These cells of the recalcitrant plant and/or regenerative plant may optionally be protoplasts, or part of a callus or stem slices (or junction slices). Similarly, the transgene or mutation can be introduced in one or more cells of the recalcitrant plant and cells of the recalcitrant plant carrying the transgene or mutation may be grafted onto cells of the, optionally non-transformed or non-mutated, regenerative plant.

Alternatively or in addition, the transgene or mutation may be introduced into one or more cells of the recalcitrant plant after contacting the one or more cells of the recalcitrant plant with one or more cells of the regenerative plant. The transgene or mutation is preferably introduced before shoot formation. Thus preferably, the step of introducing a mutation is prior to step (b2) of the method as defined herein, but may be during or after step (a) or (b1).

As a non-limiting example, the one or more cells of the recalcitrant plant may be contacted, or co-cultured, with one or more cells of the regenerative plant in vitro, followed by introducing a transgene or mutation into the one or more cells of the recalcitrant plant. Optionally, the contacted or “co-cultured” cells are first allowed to form callus, prior to introducing a transgene or mutation in at least one or more cells of the recalcitrant plant. The transgene or mutation may also be introduced into one or more cells of the regenerative plant.

Similarly, one or more cells of the recalcitrant plant may be grafted onto one or more cells of the regenerative plant, followed by introducing a transgene or mutation in a sequence of interest in one or more cells of the recalcitrant plant. The transgene or mutation may also be introduced into one or more cells of the regenerative plant. Optionally the grafted sections are first healed, prior to introducing a transgene or mutation in at least one or more cells of the recalcitrant plant. Optionally, the graft union is first cut or “wounded” prior to introducing a transgene or mutation in at least one or more cells of the recalcitrant plant. Optionally, the contacted cells are first allowed to form callus, prior to introducing a transgene or mutation in at least one or more cells of the recalcitrant plant.

Preferably, the shoot selected in step (c) of the method of the invention comprises a transgene or mutation in the sequence of interest in a cell regenerated from the cell of the recalcitrant plant of step (a) of the method of the invention. The shoot preferably comprises the transgene or mutation in at least one of the L1-, L2- and L3-shoot meristem layer regenerated from the recalcitrant cell of step (a) of the method of the invention. Preferably, at least the germline progenitor cells and/or the clonally propagated tissue and/or plant part of the selected shoot are regenerated from the recalcitrant cell of step (a) of the method of the invention and comprise a transgene or mutation in a gene of interest. Hence, a preferred method of the invention is a method of generating and selecting a shoot of a plant, wherein the shoot comprises germline progenitor cells and/or comprises cells giving rise to a clonally propagated tissue and/or plant part of a recalcitrant plant and wherein the one or more of the germline progenitor cells and/or clonally propagated plant tissue and/or plant parts comprise a transgene or mutation in a sequence of interest. Preferably, all or substantially all germline progenitor cells and/or clonally propagated tissues and/or plant parts comprise the transgene or mutation in the sequence of interest.

The transgene or mutation may be present in at least the L2-shoot meristem layer. Hence, a preferred method of the invention is a method of generating and selecting a shoot of a plant, wherein the shoot comprises an L2-shoot meristem layer of a recalcitrant plant and wherein the one or more cells of the L2-shoot meristem layer comprises a transgene or mutation in a sequence of interest. Preferably, all or substantially all cells of at least the L2-shoot meristem layer comprises the transgene or mutation in a sequence of interest. Optionally, the transgene or mutation is present in all or substantially all cells of the generated shoot selected in step (c), wherein said cells are cells regenerated from the recalcitrant plant cell of step (a) of the method of the invention.

An introduction of a transgene or a mutation in a sequence of interest in the method of the invention preferably results in a one or more improved phenotypic properties, such as but not limited to an increased yield, disease resistance, agronomic traits, abiotic traits, protein composition, oil composition, starch composition, insect resistance, fertility, silage, and morphological traits.

The transgene may be introduced by stable or transgenic transfection using any method known by the person skilled in the art to transfect a plant, plant part, callus, plant cell or protoplast.

A mutation is to be understood herein as an alteration in genetic code either in nucleotide sequence (insertion, deletion or substitution of one or more nucleotides) or epigenetic alterations such as a change in methylation. A mutation may be introduced by random mutagenesis or targeted mutagenesis, the latter also being referred to as programmed genome editing. Random mutagenesis may be, but is not limited to, chemical mutagenesis and gamma radiation. Non-limiting examples of chemical mutagenesis include, but are not limited to, EMS (ethyl methanesulfonate), MMS (methyl methanesulfonate), NaN3 (sodium azide) D), ENU (N-ethyl-N-nitrosourea), AzaC (azacytidine) and NQO (4-nitroquinoline 1-oxide). Optionally, mutagenesis systems such as TILLING (Targeting Induced Local Lesions IN Genomics; McCallum et al., 2000, Nat Biotech 18:455, and McCallum et al. 2000, Plant Physiol. 123, 439-442, both incorporated herein by reference) may be used to generate a mutation in a cell of a recalcitrant plant. TILLING uses traditional chemical mutagenesis (e.g. EMS mutagenesis) followed by high-throughput screening for mutations. Thus, plants, seeds and tissues comprising a gene having one or more of the desired mutations may be obtained using TILLING. Preferably, plants, seeds and tissues comprising a gene having one or more of the desired mutations may be obtained using KeyPoint® Breeding as described in WO2007/037678, which is incorporated herein by reference.

Targeted mutagenesis or programmed genome editing is mutagenesis that can be designed to alter a specific nucleotides or nucleic acid sequence, such as but not limited to, oligo-directed mutagenesis, RNA-guided endonucleases (e.g. the CRISPR-technology), TALENs, meganucleases or Zinc finger technology.

Preferably, the targeted mutagenesis is introduced by a site-specific protein, preferably a site-specific endonuclease. The site-specific endonuclease is preferably at least one of a CRISPR-protein complexed with a guide RNA, a TALEN, a Zinc Finger Protein, a meganuclease and an Argonaute complex. Preferably, the site-specific endonuclease is a CRISPR protein complexed with a guide RNA.

The CRISPR-protein that is part of the CRISPR protein complex for use in the method of the invention is preferably at least one of a CRISPR-endonuclease, CRISPR-nickase and a CRISPR-deaminase. Preferably, the CRISPR-protein is a CRISPR-endonuclease.

The CRISPR-protein can be any suitable CRISPR-protein known in the art. Optionally, the CRISPR-protein comprises a nuclear localisation signal (NLS) to direct the CRISPR-protein to the nucleus of the plant cell. Any known nuclear localisation signal would be suitable for use in the invention. Preferred nuclear localisation signals include, but are not limited to the NLS of the SV40 Large T-antigen MEDPTMAPKKKRKV (SEQ ID NO: 1) and the NLS of nucleoplasmin KRPAATKKAGQAKKKK (SEQ ID NO: 2).

A CRISPR-endonuclease comprises a nuclease domain and at least one domain that interacts with a guide RNA. When complexed with a guide RNA, the CRISPR protein complex is directed to a specific nucleic acid sequence by a guide RNA. The guide RNA interacts with the CRISPR-endonuclease as well as with a target-specific nucleic acid sequence, such that, once directed to the site comprising the target nucleic acid sequence via the guide sequence, the CRISPR-endonuclease is able to introduce a double-stranded break at the target site.

In case the CRISPR-protein is a CRISPR-endonuclease, both domains of the nuclease are catalytically active and the protein is able to introduce a double-stranded break at the target site. In case the CRISPR-protein is a CRISPR-nickase, one domain of the nuclease is catalytically active and one domain is catalytically inactive, and the protein is able to introduce a single-stranded break at the target site.

The skilled person is well aware of how to design a guide RNA in a manner that it, when combined with a CRISPR-endonuclease or CRISPR-nickase, effects the introduction of a single- or double-stranded break at a predefined site in the nucleic acid molecule.

CRISPR-proteins can generally be categorized into six major types (Type I-VI), which are further subdivided into subtypes, based on core element content and sequences (Makarova et al, 2011, Nat Rev Microbiol 9:467-77 and Wright et al, 2016, Cell 164(1-2):29-44). In general, the two key elements of a CRISPR-protein complex is a CRISPR-protein and a guide RNA.

Type II CRISPR-protein complexes include a signature Cas9 protein, a single protein (about 160 KDa), capable of specifically cleaving duplex DNA. The Cas9 protein typically contains two nuclease domains, a RuvC-like nuclease domain near the amino terminus and the HNH (or McrA-like) nuclease domain near the middle of the protein. Each nuclease domain of the Cas9 protein is specialized for cutting one strand of the double helix (Jinek et al, 2012, Science 337 (6096): 816-821). The Cas9 protein is an example of a CAS protein of the type II CRISPR-CAS protein complex and forms an endonuclease, when combined with the crRNA and a second RNA termed the trans-activating crRNA (tracrRNA). The crRNA and tracrRNA function together as the guide RNA. The CRISPR-protein complex introduces DNA double strand breaks (DSBs) at the position in the genome defined by the crRNA. Jinek et al. (2012, Science 337:816-820) demonstrated that a single chain chimeric guide RNA (herein defined as a “sgRNA” or “single guide RNA”) produced by fusing an essential portion of the crRNA and tracrRNA was able to form a functional CRISPR-protein complex in combination with the Cas9 protein.

A Type V CRISPR-protein complex has been described, the Clustered Regularly Interspaced Short Palindromic Repeats from Prevotella and Francisella 1 or CRISPR/Cpf1. Cpf1 genes are associated with the CRISPR locus and coding for an endonuclease that use a crRNA to target DNA. Cpf1 is a smaller endonuclease than Cas9, which may overcome some of the CRISPR-Cas9 system limitations. Cpf1 is a single RNA-guided endonuclease lacking tracrRNA, and it utilizes a T-rich protospacer-adjacent motif. Cpf1 cleaves DNA via a staggered DNA double-stranded break (Zetsche et al (2015) Cell 163 (3):759-771). The type V CRISPR-Protein system preferably includes at least one of Cpf1, C2c1 and C2c3.

The CRISPR-protein complex for use in the invention may comprise any CRISPR-protein as defined herein above. Preferably, the CRISPR-protein is a Type II CRISPR-protein, preferably a Type II CRISPR-endonuclease, e.g., Cas9 (e.g., the protein of SEQ ID NO: 3, encoded by SEQ ID NO: 4, or the protein of SEQ ID NO: 5) or a Type V CRISPR-protein, preferably a Type V CRISPR-endonuclease, e.g. Cpf1 (e.g., the protein of SEQ ID NO: 6, encoded by SEQ ID NO: 7) or Mad7 (e.g. the protein of SEQ ID NO: 8 or 9), or a protein derived thereof, having preferably at least about 70%, 80%, 90%, 91%, 92%, 93%, 94%, 95%, 96%, 97%, 98%, or 99% sequence identity to said protein over its whole length.

Preferably, the CRISPR-protein is a Type II CRISPR-endonuclease, preferably a Cas9 endonuclease.

The skilled person knows how to find and prepare a CRISPR-protein for use in the method of the invention. In the prior art, numerous reports are available on its design and use. See for example the review by Haeussler et al (J Genet Genomics. (2016)43(5):239-50. doi:10.1016/j.jgg.2016.04.008.) on the design of guide RNA and its combined use with a CAS-protein (originally obtained from S. pyogenes), or the review by Lee et al. (Plant Biotechnology Journal (2016) 14(2) 448-462).

In general, a CRISPR-endonuclease, such as Cas9, comprises two catalytically active nuclease domains. For example, a Cas9 protein can comprise a RuvC-like nuclease domain and an HNH-like nuclease domain. The RuvC and HNH domains work together, both cutting a single strand, to make a double-stranded break in DNA. (Jinek et al., Science, 337:816-821).

A dead CRISPR-endonuclease comprises modifications such that none of the nuclease domains shows cleavage activity. The CRISPR-nickase may be a variant of the CRISPR-endonuclease wherein one of the nuclease domains is mutated such that it is no longer functional (i.e., the nuclease activity is absent). An example is a SpCas9 variant having either the D10A or H840A mutation.

The CRISPR-protein may comprise or consist of a whole type II or type V CRISPR-protein or a variant or functional fragment thereof. Preferably such fragment binds the guide RNA and maintains, at least partly, endonuclease activity.

Preferably, the CRISPR-protein for use in the method of the invention is a Cas9 protein. The Cas9 protein may be derived from the bacteria Streptococcus pyogenes (SpCas9; NCBI Reference Sequence NC_017053.1; UniProtKB-Q99ZW2), Geobacillus thermodenitrificans (UniProtKB-A0A178TEJ9), Corynebacterium ulcerous (NCBI Refs: NC_015683.1, NC_017317.1); Corynebacterium diphtheria (NCBI Refs: NC_016782.1, NC_016786.1); Spiroplasma syrphidicola (NCBI Ref: NC_021284.1); Prevotella intermedia (NCBI Ref: NC_017861.1); Spiroplasma taiwanense (NCBI Ref: NC_021846.1); Streptococcus iniae (NCBI Ref: NC_021314.1); Belliella baltica (NCBI Ref: NC_018010.1); Psychroflexus torquisl (NCBI Ref: NC_018721.1); Streptococcus thermophilus (NCBI Ref: YP_820832.1); Listeria innocua (NCBI Ref: NP_472073.1); Campylobacter jejuni (NCBI Ref: YP_002344900.1); or Neisseria meningitidis (NCBI Ref: YP_002342100.1). Encompassed are Cas9 variants from these, having an inactivated HNH or RuvC domain homologues to SpCas9,, e.g. the SpCas9_D10A or SpCas9_H840A, or a Cas9 having equivalent substitutions at positions corresponding to D10 or H840 in the SpCas9 protein, rendering a nickase.

The CRISPR-protein for use in the method of the invention may be, or may be derived from, Cpf1, e.g. Cpf1 from Acidaminococcus sp; UniProtKB-U2UMQ6. The variant may be a Cpf1-nickase having an inactivated RuvC or NUC domain, wherein the RuvC or NUC domain has no nuclease activity anymore. The skilled person is well aware of techniques available in the art such as site-directed mutagenesis, PCR-mediated mutagenesis, and total gene synthesis that allow for inactivated nucleases such as inactivated RuvC or NUC domains. An example of a Cpf1 nickase with an inactive NUC domain is Cpf1 R1226A (see Gao et al. Cell Research (2016) 26:901-913, Yamano et al. Cell (2016) 165(4): 949-962). In this variant, there is an arginine to alanine (R1226A) conversion in the NUC-domain, which inactivates the NUC-domain.

The CRISPR-protein for use in the method of the invention may be, or may be derived from, CRISPR-Casϕ, a nuclease that is about half the size of Cas9. CRISPR-Casϕ uses a single crRNA for targeting and cleaving the nucleic acid as is described e.g. in Pausch et al (CRISPR-Casϕ from huge phages is a hypercompact genome editor, Science (2020); 369(6501):333-337).

An active, partly inactive or dead CRISPR-protein may be used in the method of the invention, e.g. to guide a fused functional domain as detailed herein to a specific site in the DNA as determined by the guide RNA.

Hence, the CRISPR-protein may be fused to a functional domain. Optionally, such functional domain is for epigenetic modification, for example a histone modification domain. The domains for epigenetic modification can be selected from the group consisting of a methyltransferase, a demethylase, a deacetylase, a methylase, a deacetylase, a deoxygenase, a glycosylase and an acetylase (Cano-Rodriguez et al, Curr Genet Med Rep (2016) 4:170-179). The methyltransferase may be selected from the group consisting of G9a, Suv39h1, DNMT3, PRDM9 and Dot1L. The demethylase may be LSD1.The deacetylase may be SIRT6 or SIRT3. The methylase may be at least one of KYP, TgSET8 and NUE. The deacetylase may be selected from the group consisting of HDAC8, RPD3, Sir2a and Sin3a. The deoxygenase may be at least one of TET1, TET2 and TET3, preferably TET1cd (Gallego-Bartolomé J et al, Proc Natl Acad Sci U S A. (2018); 115(9):E2125-E2134). The glycosylase may be TDG. The acetylase may be p300.

Optionally, the functional domain is a deaminase, or functional fragment thereof, selected from the group consisting of an apolipoprotein B mRNA-editing complex (APOBEC) family deaminase, an activation-induced cytosine deaminase (AID), an ACF1/ASE deaminase, an adenine deaminase, and an ADAT family deaminase. Alternatively or in addition, the deaminase or functional fragment thereof may be ADAR1 or ADAR2, or a variant thereof.

The apolipoprotein B mRNA-editing complex (APOBEC) family of cytosine deaminase enzymes encompasses eleven proteins that serve to initiate mutagenesis in a controlled and beneficial manner. Preferably, the APOBEC deaminase is selected from the group consisting of APOBEC1, APOBEC2, APOBEC3A, APOBEC3B, APOBEC3C, APOBEC3D, APOBEC3F, APOBEC3G, APOBEC3H, APOBEC4 and Activation-induced (cytidine) deaminase. Preferably, the cytosine deaminase of the APOBEC family is activation-induced cytosine (or cytidine) deaminase (AID) or apolipoprotein B editing complex 3 (APOBEC3). Preferably, the deaminase domain fused to the CRISPR-protein an APOBEC1 family deaminase.

Another exemplary suitable type of deaminase domain that may be fused to the CRISPR-system nuclease is an adenine or adenosine deaminase, for example an ADAT family of adenine deaminase. Further, the adenine deaminase may be TadA or a variant thereof, preferably as described in Gaudelli et al., 2017 (Gaudelli et al. 2017 Nature 551: 464-471). Further, the CRISPR-system nuclease may be fused to an adenine deaminase domain, e.g. derived from ADAR1 or ADAR2. The deaminase domain of the present invention may comprise or consist of a whole deaminase protein or a fragment thereof which has catalytic activity. Preferably, the deaminase domain has deaminase activity. Optionally, the CRISPR-protein is further fused to an UDG inhibitor (UGI) domain.

The CRISPR-protein for use in the method of the invention is complexed with a guide RNA molecule, which guides the CRISPR-protein to a specific location in the genome of a plant cell to achieve a targeted genomic modification. Preferably the plant cell is a cell of a recalcitrant plant. Optionally, the plant cell is a germline or germline progenitor cell and/or a cell giving rise to a clonally propagated tissue and/or plant part of a recalcitrant plant.

The complex comprising a CRISPR-protein and a guide RNA may also be annotated as a ribonucleoprotein complex.

The guide RNA molecule directs the complex to a defined target site in a double-stranded nucleic acid molecule, also named the protospacer sequence. The guide RNA molecule comprises a sequence for targeting the CRISPR-protein complex to a protospacer sequence that is preferably near, at or within a sequence of interest in the genome of the plant cell. The guide RNA can be a single guide (sg)RNA or the combination of a crRNA and a tracrRNA (e.g. for Cas9) or a crRNA only (e.g. in case of Cpf1 and Casϕ).

The CRISPR-protein complex for use in the method of the invention may thus comprise a guide RNA molecule, wherein the guide RNA molecule comprises a combination of a crRNA and a tracrRNA, and wherein preferably the CRISPR-protein is Cas9. The crRNA and tracrRNA are preferably combined into a sgRNA (single guide RNA). Alternatively, the CRISPR-protein complex for use in the method of the invention may comprise a guide RNA molecule, wherein the guide RNA molecule comprises a crRNA, and wherein preferably the CRISPR protein is Cpf1 or Casϕ.

The guide RNA molecule for use in a method of the invention may comprise a sequence that can hybridize to or near a sequence of interest, preferably a sequence of interest as defined herein. The guide RNA molecule may comprise a nucleotide sequence that is fully complementary to a sequence in the sequence of interest, i.e. the sequence of interest comprises a protospacer sequence. Alternatively or in addition, the guide RNA molecule for use in the method of the invention may comprise a sequence that can hybridize to or near the complement of a sequence of interest.

The part of the crRNA that is complementary to the protospacer sequence is designed to have sufficient complementarity with the protospacer sequence to hybridize with the protospacer sequence and direct sequence-specific binding of a complexed CRISPR protein. The protospacer sequence is preferably adjacent to a protospacer adjacent motif (PAM) sequence, which PAM sequence may interact with the CRISPR protein of the RNA-guided CRISPR-protein complex. For instance, in case the CRISPR protein is S. pyogenes Cas9, the PAM sequence preferably is 5′-NGG-3′, wherein N can be any one of T, G, A or C.

The skilled person is capable of engineering the crRNA to target any desired sequence, preferably by engineering the sequence to be at least partly complementary to any desired protospacer sequence, in order to hybridize thereto. Preferably, the complementarity between part of a crRNA sequence and its corresponding protospacer sequence, when optimally aligned using a suitable alignment algorithm, is at least 70%, 80%, 85%, 90%, 91%, 92%, 93%, 94%, 95%, 96%, 97%, 98% or 100%. The part of the crRNA sequence that is complementary to the protospacer sequence may be at least about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length. In some preferred embodiments, the sequence complementary to the sequence of interest is less than about 75, 50, 45, 40, 35, 30, 25, 20 nucleotides in length. Preferably, the length of the sequence complementary to the sequence of interest is at least 17 nucleotides. Preferably the complementary crRNA sequence is about 10-30 nucleotides in length, about 17-25 nucleotides in length or about 15-21 nucleotides in length. Preferably the part of the crRNA that is complementary to the protospacer sequence is 15, 16, 17, 18, 19, 20, 21, 22, 23, 24 or 25 nucleotides in length, preferably 20 or 21 nucleotides, preferably 20 nucleotides.

Molecules suitable as crRNA and tracrRNA are well known in the art (see e.g., WO2013142578 and Jinek et al., Science (2012) 337, 816-821). The crRNA and tracrRNA in the guide RNA molecule can be linked to together to form a single guide (sg)RNA. The crRNA and tracrRNA can be linked, preferably covalently linked, using any conventional method known in the art. Covalent linkage of the crRNA and tracrRNA is e.g. described in Jinek et al. (supra) and WO13/176772, which are incorporated herein by reference. The crRNA and tracrRNA can be covalently linked using e.g. linker nucleotides or via direct covalent linkage of the 3′ end of the crRNA and the 5′ end of the tracrRNA.

Preferably, at least one CRISPR-protein complex comprising a CRISPR-nuclease and a guide RNA is used in the method of the invention. However, the skilled person straightforwardly understands that additional CRISPR-protein complexes can be used in the method of the invention, e.g. by the use of at least 2, 3, 4, 5, 6, 7, 8, 9, 10 or more different guide RNAs. These different guide RNAs van be designed to target and bind to the same sequence of interest. Alternatively, different guide RNAs may direct the CRISPR-protein complex to different genes of interest.

The transgene or mutation in a sequence of interest may be introduced prior to callus formation, during callus formation and/or after callus formation. The transgene or mutation is preferably introduced prior to the onset of shoot formation. Preferably, the transgene or mutation is present in at least a germline progenitor cell and/or clonally propagated tissue and/or plant part of a shoot formed in step b) of the method of the invention. Preferably, the transgene or mutation is present in at least a germline progenitor cell and/or present in a cell giving rise to a clonally propagated tissue and/or plant part of a shoot selected in step c). Preferably, the transgene or mutation is present in all germline progenitor cells and/or all clonally propagated tissues and/or plant parts of a shoot formed in step b). Preferably, the transgene or mutation is present in at least one cell of the L2-shoot meristem layer of a shoot formed in step b) of the method of the invention. Preferably, the transgene or mutation is present in all cells of the L2-shoot meristem layer of a shoot formed in step b). The transgene or mutation may also be present in other cells, such as cells of the L1- and L3-shoot meristem layer. Optionally, all cells of a shoot formed in step c) of the method of the invention comprise the transgene or mutation in a sequence of interest.

The transgene or mutation in a sequence of interest may be introduced in a cell of a recalcitrant plant and/or in a cell of a regenerative plant. Preferably, the transgene or mutation in a sequence of interest is at least introduced in the cell of a recalcitrant plant of step (a) of the method of the invention and/or in a cell of a recalcitrant plant co-regenerated in step (b) of the method of the invention.

The mutation may be introduced by transfecting the plant cell with a site-specific endonuclease, preferably a CRISPR-endonuclease. The transgene may be introduced by transfecting the plant cell with a transgene of interest. Transfection of a plant cell can be performed using any conventional means known to the person skilled in the art.

“Transfection” or “transformation” is understood herein as the delivery of a transgene and/or site-specific endonuclease protein or a nucleic acid molecule encoding the transgene and/or site-specific endonuclease into the plant cell. Said nucleic acid molecule may be DNA or RNA encoding said transgene and/or site-specific nuclease. Optionally the transgene and/or site-specific endonuclease is introduced by transfection of (pre-)mRNA. Transfection may further include the delivery of a guide RNA or a nucleic acid molecule encoding the guide RNA (to be) associated with a site-specific endonuclease into the plant cell. Optionally, the site-specific endonuclease is delivered as a CRISPR-endonuclease complex comprising a CRISPR-endonuclease complexed with a guide RNA. Alternatively or in addition, the CRISPR-endonuclease and the guide RNA are delivered into the plant cell, and form a complex intracellularly. Alternatively or in addition, the CRISPR-endonuclease is expressed from the transfected nucleic acid and forms intracellularly a complex with the, optionally expressed, guide RNA.

Preferably the transgene and/or site-specific endonuclease, or nucleic acid encoding the same, may be introduced as a protein, or in case of a CRISPR endonuclease as a protein-guide RNA complex (also called a ribonucleoprotein complex), into a cell of a recalcitrant plant using any conventional means known by the skilled person. Non-limiting examples of transfection include, but are not limited to, viral infection, conjugation, protoplast fusion, electroporation, particle gun technology, calcium phosphate precipitation, direct microinjection, silicon carbide whiskers technology, Agrobacterium-mediated transformation and the like. The choice of method is generally dependent on the type of cell being transformed and the circumstances under which the transformation is taking place (i.e. in vitro, ex vivo, or in vivo; protein transfection or nucleic acid transfection).

Transfection methods based upon the soil bacterium Agrobacterium tumefaciens may be particularly useful for introducing the nucleic acid molecule into a plant cell. Methods of co-culturing Agrobacterium with cultured plant cells or wounded tissue such as leaf tissue, root explants, hypocotyledons, stem pieces or tubers, for example, are well known in the art. See., e.g., Glick and Thompson, (eds.), Methods in Plant Molecular Biology and Biotechnology, Boca Raton, Fla.: CRC Press (1993). Microprojectile-mediated transformation also can be used to transfect the plant cell. This method, first described by Klein et al. (Nature 327:70-73 (1987)), relies on microprojectiles such as gold or tungsten that are coated with e.g. the desired nucleic acid molecule by precipitation with calcium chloride, spermidine or polyethylene glycol. The microprojectile particles are accelerated at high speed into an angiosperm tissue using a device such as the BIOLISTIC PD-1000 (Biorad; Hercules Calif.).

A nucleic acid encoding the transgene and/or the site-specific endonuclease, and optionally a (nucleic acid encoding) a guide RNA, may be introduced into a plant in a manner such that the nucleic acid is able to enter a plant cell(s), e.g., via an in vivo or ex vivo protocol. By “in vivo,” it is meant in the nucleic acid is administered to a living body of a plant e.g. infiltration. By “ex vivo” it is meant that cells or explants are modified outside of the plant, and then such cells or organs are regenerated into a shoot of a plant.

A number of vectors suitable for transformation of plant cells and/or for the establishment of transgenic plants have been described, including those described in Weissbach and Weissbach, (1989) Methods for Plant Molecular Biology Academic Press, and Gelvin et al., (1990) Plant Molecular Biology Manual, Kluwer Academic Publishers. Examples include Agrobacterium tumefaciens-mediated transformation, as well as those methods e.g. disclosed by Herrera-Estrella et al. (1983) Nature 303:209, Bevan (1984) Nucl Acid Res. 12:8711-8721, Klee (1985) Bio/Technology 3:637-642. Conventional methods for transforming a plant cell include, but is not limited to, biolistic bombardment, polyethylene glycol transformation, and microinjection (see e.g. Danieli et al Nat.Biotechnol 16:345-348, 1998; Staub et al Nat. Biotechnol 18:333-338, 2000; O'Neill et al Plant J. 3:729-738, 1993; Knoblauch et al Nat. Biotechnol 17:906-909; U.S. Pat. Nos. 5,451,513, 5,545,817, 5,545,818, and 5,576,198; in Intl. Application No. WO 95/16783; and in Boynton et al., Methods in Enzymology 217:510-536 (1993), Svab et al., Proc. Natl. Acad. Sci. USA 90:913-917 (1993), and McBride et al., Proc. Nati. Acad. Sci. USA 91:7301-7305 (1994).

Preferably, the transgene is introduced in a cell of the recalcitrant plant and/or the mutation is in a sequence of interest in a cell of the recalcitrant plant. A cell of the recalcitrant plant is preferably transfected with at least one of a transgene, a CRISPR endonuclease and/ a one guide RNA. Preferably, the CRISPR-endonuclease and the guide RNA form a ribonucleoprotein complex that is transfected into the cell of the recalcitrant plant. Preferably, said cell is a protoplast. Preferably the protoplast is transfected with a transgene protein and/or a CRISPR-guide RNA ribonucleoprotein complex using polyethylene glycol transformation, e.g. such as described in WO2017/222370 or WO2020/089448, which are incorporated herein by reference. The cell may be a cell in a single cell suspension, a protoplast, a cell present in a callus or a slice, and/or a cell present in a plant, preferably present in a graft union.

Alternatively or in addition, a cell of the recalcitrant plant may be transfected with a nucleic acid molecule encoding the transgene and/or at least one site-specific endonuclease and/or at least one guide RNA. Optionally, said cells is a protoplast. Optionally, the protoplast is transfected with one or more plasmids encoding the transgene and/or CRISPR-endonuclease and a guide RNA using polyethylene glycol transformation, e.g. such as described in WO2018/115390 and WO/2020/011985, which are incorporated herein by reference.

Preferably the codon sequence of the transgene and/or site-specific endonuclease is optimized for expression in plant cells. The nucleic acid molecule encoding at least one transgene and/or site-specific endonuclease and/or at least one guide RNA is preferably comprised in a nucleic acid vector. The nucleic acid vector is preferably a vector for transient expression of the transgene and/or site-specific endonuclease and/or guide RNA. Alternatively, the nucleic acid vector is a vector for stable expression of the transgene and/or site-specific endonuclease and/or guide RNA. Optionally, a cell of the recalcitrant plant of step a) of the method of the invention comprises a transgene integrated in its genome that encodes for a gene or interest and/or a programmable endonuclease, preferably for a CRISPR endonuclease, wherein said transgene and/or programmable endonuclease may be stably expressed or wherein the expression of said transgene and/or programmable endonuclease is under the control of an inducible or tissue specific promoter.

The transgene and/or site-specific endonuclease and optionally at least one guide RNA may thus be transcribed from an expression cassette comprised in the vector. The vector backbone may for example be a plasmid into which the expression cassette is integrated or, if a suitable transcription regulatory sequence is already present (for example a (inducible) promoter), only a desired nucleotide sequence (e.g. a sequence encoding the transgene and/or site-specific endonuclease) is integrated downstream of the transcription regulatory sequence.

The vector for use in the method of the invention may comprise further genetic elements to facilitate their use in molecular cloning, such as e.g. selectable markers, multiple cloning sites and the like. The vector backbone may for example be a binary or superbinary vector (see e.g. U.S. Pat. No. 5,591,616, US 2002138879 and WO 95/06722), a co-integrate vector or a T-DNA vector, as known in the art.

Vectors for use in the method of the invention are preferably particularly suitable for introducing the expression of a transgene and/or site-specific endonuclease and optionally one or more guide RNAs into a plant cell, wherein the plant cell is preferably a recalcitrant plant cell. A preferred expression vector is a naked DNA, a DNA complex or a viral vector.

A preferred naked DNA is a linear or circular nucleic acid molecule, e.g. a plasmid. A plasmid refers to a circular double stranded DNA loop into which additional DNA segments can be inserted, such as by standard molecular cloning techniques. A DNA complex can be a DNA molecule coupled to any carrier suitable for delivery of the DNA into the cell. A preferred carrier is selected from the group consisting of a lipoplex, a liposome, a polymersome, a polyplex, PEG, a dendrimer, an inorganic nanoparticle, a virosome and cell-penetrating peptides.

The vector for use in the method of the invention is preferably a viral expression vector. The viral vector van be an DNA virus or an RNA virus. The viral vector may be, or may be based on, a Tobamovirus, a Tobravirus, a Potexvirus, a Geminivirus, an Alfamovirus, a Cucumovirus, a Potyvirus, a Tombusvirus, a Hordeivirus, or a Nucleorhabdovirus.

The Tobamovirus viral vector may be at least one of a Tobacco Mosaic Virus (TMV) and a Sun Hemp Mosaic Virus (SHMV). The Tobravirus viral vector may be a Tobacco Rattle Virus (TRV). The Potex virus viral vector may be at least one of Potatovirus X (PVX) and the papaya mosaic potexvirus (PapMV). The Geminivirus viral vector may be a Comovirus Cowpea mosaic virus (CPMV). Further examples of suitable Geminivirus viral vectors may include the cabbage leaf curl virus, tomato golden mosaic virus, bean yellow dwarf virus, African cassava mosaic virus, wheat dwarf virus, miscanthus streak mastrevirus, tobacco yellow dwarf virus, tomato yellow leaf curl virus, bean golden mosaic virus, beet curly top virus, maize streak virus, and tomato pseudo-curly top virus. The Alfamovirus may an alfalfa mosaic virus (AMV). The Cucumovirus may be a cucumber mosaic virus (CMV). The Potyvirus may be a plum pox virus (PPV). The Tombusvirus may be a tomato bushy stunt virus (TBSV). The Hordeivirus may be a barley stripe mosaic virus. The Nucleorhabdovirus may be a Sonchus Yellow Net Virus (SYNV) (see e.g. Hefferon K, Plant Virus Expression Vectors: A Powerhouse for Global Health, Biomedicines. 2017, 5(3):44 and Lico et al, Viral vectors for production of recombinant proteins in plants, J Cell Physiol, 2008;216(2):366-77).

Preferably, the viral vector is selected from the group consisting of a Tobacco Rattle Virus (TRV), Tobacco Mosaic Virus (TMV), a Sonchus Yellow Net Virus (SYNV) and Potato Virus X (PVX). Preferably, the viral vector is at least one of a Tobacco Rattle Virus (TRV), a Tobacco Mosaic Virus (TMV) and a Sonchus Yellow Net Virus (SYNV). The viral vector for use in the method of the invention may comprise a deletion of a gene to increase the packaging capacity of the virus. Preferably, the virus comprises a deletion of a gene encoding the coat protein (CP). A preferred viral vector comprising a deletion of the coat protein is a Tobamovirus virus or a Tobravirus virus. Preferably the viral vector comprising a deletion of a coat protein is a Tobamovirus, preferably the Tobacco Mosaic Virus (TMV). A preferred viral vector is the TMV RNA-based overexpression vector (TRBO), e.g. as described in Lindbo (TRBO: A High-Efficiency Tobacco Mosaic Virus RNA-Based Overexpression Vector, Plant Physiol, 2007; 145(4):1232-40). The viral vector may be a self-replicating RNA as e.g. described in WO2018/226972, which is incorporated herein by reference.

The vector, preferably the viral vector, may be comprised in an Agrobacterium to initially introduce the viral vector into a plant cell of the plant. After infection, the viral vector is expressed from the Agrobacterium in the plant cell. The viral vector may replicate and infect surrounding plant cells. The viral vector may be modified, e.g. by deletion of the coat protein, which prevents systemic spread of the virus.

The cell of the recalcitrant plant that is transfected will preferably develop into a tissue that is part of a newly formed shoot, wherein the tissue comprises one or more germline progenitor cells and/or one or more cells giving rise to a clonally propagated tissue and/or plant part. The transfected recalcitrant cell may be a primary transfected cell, or e.g. a secondary or subsequently transfected cell. As a non-limiting example, a cell of a regenerative plant may be transfected with a vector, such as e.g. an agrobacterium and/or a viral vector, expressing a transgene and/or a site-specific endonuclease. The virus produced in these initially infected cells may spread and infect the cell of a recalcitrant plant, i.e. in a secondary infection.

For example, one or more cells of the regenerative plant may be infected with an agrobacterium comprising a viral vector expressing a transgene and/or a site-specific endonuclease. In the graft union, the produced virus may translocate to cells of the recalcitrant plant. Subsequent infection of the viral vector results in expression of a transgene and/or a site-specific endonuclease in one or more cells of the recalcitrant plant. The site-specific endonuclease will introduce a mutation in a sequence of interest in the recalcitrant cell and upon shoot formation, the mutation will be present in the formed shoots. The transgene may be integrated in the recalcitrant plant upon shoot formation and the transgene may be present in the formed shoots.

As indicated above, the method of the invention may further comprise a step (d) of growing a plant from the shoot selected in step (c). Optionally, especially in case root regeneration is cumbersome, step (d) may comprise a step of grafting the selected shoot on a graft compatible rootstock. The plant grown in step (d) preferably comprises at least one inflorescence for reproduction, i.e. to produce seed and/or progeny plants.

More in particular, the invention provides for a method of generating a plant, wherein the method comprises the steps (a), (b) and (c) as defined herein and further comprising the step of generating a plant from said shoot, wherein preferably said plant comprises at least one inflorescence. Optionally, the generated plant is free or substantially free of cells of the regenerative plant of step (a). In other words, optionally the generated plant is a non-chimera plant having the same or substantially the same genotype of the recalcitrant plant of step (a) of the method of the invention. Hence, the generated plant may also be a recalcitrant plant as defined herein, preferably of the same species and variety as the recalcitrant plant cell of step (a). In case the method of the invention comprises the introduction of a mutation and/or transgene, preferably at least one of the cells, optionally all cells, of the generated plant also comprise(s) said mutation and/or transgene. Therefore, “substantially the same genotype” is to be understood herein as the same genotype albeit comprising a mutation and/or a transgene that may be introduced using a method of the invention.

In addition or alternatively, the germline cells, preferably the gametes, of the generated plant may have the same or substantially the same genotype as gametes of the recalcitrant plant of step (a) of the method of the invention, optionally comprising a mutation and/or transgene introduced in the cell of the recalcitrant plant of step (a) of the method of the invention as further detailed herein. Optionally, the plant may be a chimeric plant that further comprises cells or tissue layers of the regenerative plant. Optionally, said plant is used for producing seed and/or progeny by crossing, selfing and/or apomictic propagation in case of an apomictic genotype of the germline progenitor cells (i.e. apomictic reproduction). Optionally, the plant is pollinated and/or the pollen are used to pollinate another plant or the same plant (selfing). Optionally, said plant is used for producing a tissue and/or plant part for clonal propagation as defined herein, and optionally, said tissue and/or plant part is isolated and used clonal or vegetative propagation. Therefore, the invention also provides for a method of producing a plant or seed, comprising the steps (a), (b) and (c) as defined herein and further comprising the steps of generating a plant from the shoot selected in step (c) by vegetative or clonal propagation, wherein preferably said plant comprises at least one inflorescence; and optionally producing seed and/or a progeny plant of the generated plant by sexual or apomictic reproduction.

Hence, the method of the invention may be a method of producing a plant or seed, wherein said method comprises the steps of:
(a) contacting a cell of the recalcitrant plant with a cell of a regenerative plant;
(b) allowing the contacted cells of (a) to form shoots; and
(c) selecting a shoot formed in step (b), wherein at least part of said shoot consists of cells of the recalcitrant plant,
(d) growing a plant from the shoot selected in step (c); and optionally
(e) producing seed and/or progeny from the plant of step (d).

In case the method of the invention comprises the introduction of a mutation and/or transgene, the seed and/or progeny of the generated plant may be selected for having said mutation and/or transgene. The seed produced (or embryo of said seed) may have a genotype that is the same or substantially the same as offspring of the recalcitrant plant from which the recalcitrant plant cell of step (a) has been isolated or is part of, optionally with the exception of the introduced mutation and/or transgene.

Optionally, step (b) of regenerating a shoot from the cells contacted in step (a) comprises the formation of a callus prior to shoot regeneration. Hence, the method of the invention may be a method for producing a plant, wherein the plant comprises germline progenitor cells and/or a tissue and/or plant parts for clonal propagation of a recalcitrant plant, and wherein the method comprises the steps of:
(a) contacting one or more cells of the recalcitrant plant with one or more cells of a regenerative plant;
(b) allowing the contacted cells of the recalcitrant plant of step (a) to form callus, and allowing shoots to grow from the callus obtained in step (b);
(c) selecting a shoot obtained in step (b) comprising germline progenitor cells and/or cells giving rise to a tissue and/or plant parts for clonal propagation of the recalcitrant plant; and
(d) growing a plant from the shoot selected in step (c).

Optionally, multiple seeds and/or progeny plants are produced and the method further comprises a step of selecting at least one seed and/or progeny plant, preferably after genotyping and/or assessing the presence of the mutation and/or transgene that may have been introduced in the recalcitrant cell of step (a) of the method of the invention as detailed herein. The seed and/or progeny plant may be genotyped to assess whether the plant has the same or substantially the same genotype of the cell of the recalcitrant plant of step (a). The seed may be allowed to germinate and develop into a plant.

Optionally, the cell of the recalcitrant plant of step (a) of the method of the invention is a cell with aberrant ploidy, and may be haploid. Hence the method of the invention may be a method to propagate haploid plant material. Said method may further comprise a step of screening regenerated plants and/or seeds for ploidy levels.

Optionally, during co-regeneration in step (b) the genome may doubled spontaneously or may be doubled chemically, thereby generating at least one shoot that comprises or consists of doubled haploid cells. In that case, the genotype of the generated shoot may differ from the recalcitrant plant cell in that the genome is doubled. Hence the method of the invention may be a method to produce doubled haploid plant material, and the method of the invention may comprise a step of screening regenerated plants and/or seeds for ploidy levels.

In another embodiment, the selected shoot of step (c) is not isolated but is allowed to grow an inflorescence on the plant structure developed in step (b) of the method of the invention, wherein said plant structure optionally comprises further shoots. Said inflorescence may be used for sexual or apomictic reproduction. Said inflorescence may be pollinated or pollen of said inflorescence is used to pollinate another plant or the same plant (i.e. the inflorescence is selfed).

As indicated herein, the method may further comprise a step of introducing in the cell of the recalcitrant plant of step (a) or in a cell originating therefrom in the shoot regenerated in step (b):
(i) a transgene; or
(ii) a mutation in a sequence of interest.

Preferably, said sequence of interest is an endogenous sequence of interest. In a method comprising the introduction of a transgene or mutation, preferably, the step of introducing the transgene or the mutation is prior to step (b), and even more preferably prior to step (a).

In addition or alternatively, in said method at least a germline progenitor cell and/or cells giving rise to clonally propagating tissue and/or plant part of the shoot regenerated in step (b) comprises the transgene or the mutation. Optionally, the mutation is introduced by programmed genome editing, preferably using a site-specific endonuclease, preferably a CRISPR endonuclease.

Optionally, the cell of the recalcitrant plant in step (a) of the method of the invention is a (highly) heterogenetic, and the method of the invention is a method of propagating heterogenetic plant material. Optionally, the cell of the recalcitrant plant of step (a) is sterile, and the method of the invention is a method or propagating sterile plant material.

The plant may be grown from the shoot selected in step (c) of the method of the invention using any conventional culturing conditions known in the art by the skilled person. These culturing conditions may be dependent on the plant produced by the method of the invention and the skilled person knows how to adjust these conditions to generate an optimal environment for growing the plant produced by the method of the invention. The plant grown in step (d) may comprise a transgene or mutation in a sequence of interest as defined herein.

The method of the invention may further comprise a step (e) of producing or obtaining progeny of the plant grown in step (d). The progeny may e.g. be produced by sexual propagation, i.e. through the union of a pollen and an egg to produce a seed. Preferably, at least one of the pollen and the egg are derived from the plant produced in step (d). In case the method comprises the introduction of a transgene or mutation in a sequence of interest as defined herein, preferably, at least one of the pollen and the egg comprises the transgene or mutation in the sequence of interest. Optionally, both the pollen and egg are derived from the plant grown in step (d). Preferably, the pollen and the egg comprise the same transgene and/or mutation in the sequence of interest. Alternatively, the progeny is obtained by a-sexual (vegetative) propagation of the plant grown in step (d). Preferably, within such embodiment, the transgene and/or mutation in the sequence of interest is present in the tissue and/or plant part that is clonally propagated to form the next generation.

The invention also pertains to a plant obtainable by the method of the invention, preferably in step (d) by the method of the invention. The plant may be a chimera plant comprising cells having the same or substantially the same genotype of the recalcitrant plant and cells or tissues having the same or substantially the same genotype of the regenerative plant. Preferably, the plant comprises germline or germline progenitor cells and/or a tissue and/or plant part for clonal propagation of the recalcitrant plant. Preferably, the plant comprises an L2-shoot meristem layer of the recalcitrant plant. Optionally the plant is a periclinal chimera and/or a plant, preferably a recalcitrant plant or a periclinal chimera, comprising a transgene or mutation in a sequence of interest. Hence, the plant may be a non-natural plant, a man-made plant, a mutant plant and/or a transformed plant.

In an aspect, the invention thus concerns a periclinal chimera obtainable from the method of the invention, preferably obtainable from step (d) as defined herein. “Periclinal chimeras” are chimeras in which one or more entire cell (tissue) layer(s) L1, L2, and/or L3 is genetically distinct from another cell layer. In the case of periclinal chimeras, a single tissue layer itself is homogeneous and not chimeric. Periclinal chimeras are the most stable forms of chimeras, and produce distinctive and valuable plant phenotypes. These plants produce axillary buds that possess the same apical organization as the terminal meristem from which they were generated. Therefore, periclinal chimeras can be multiplied by vegetative propagation and maintain their chimera layer organization.

The periclinal chimera plant obtainable from the method of the invention preferably comprises at least one shoot meristem layer of the recalcitrant plant and at least one shoot meristem layer of the regenerative plant. Preferably at least one of the L1-, L2- and L3-shoot meristem layer is from a recalcitrant plant. The shoot meristem layer that is not from the recalcitrant plant is preferably from a regenerative plant. Preferably, the L2-shoot meristem layer of the periclinal chimera is of a recalcitrant plant and at least one of the L1- and L3-shoot meristem layer is of the regenerative plant.

The L2-meristem layer and the L1- and L3-shoot meristem layer of the periclinal plant can be of the same or of a different genus. Preferably, the L2-meristem layer and the L1- and L3-shoot meristem layer of the periclinal plant are of the same genus. As a non-limiting example, the L1-, L2- and L3-shoot meristem layer can be of the genus Solanum or of the genus Capsicum. For example, the L2-shoot meristem layer can be from a Capsicum annuum plant and at least one of the L1- and L3-shoot meristem layer can be from a Capsicum baccatum plant. Similarly, the L2-shoot meristem layer can be from a Solanum tuberosum plant and at least one of the L1- and L3-shoot meristem layer can be from a Solanum lycopersicum plant.

The periclinal chimera may further comprise a transgene or a mutation in a sequence of interest. The mutation is preferably present in at least a germline or germline progenitor cell and/or a tissue and/or plant part for clonal propagation of the recalcitrant plant. Preferably, the transgene or mutation is in a cell located in at least one of the L1-, L2- and L3-shoot meristem layer of the periclinal chimera. Preferably, the transgene or mutation is present in a cell located in at least the L2-shoot meristem layer of the periclinal chimera.

In a further aspect, the invention pertains to a plant obtainable from the method of the invention, wherein the plant comprises a transgene and/or mutation in a sequence of interest. Hence the plant may be a transgenic plant and/or mutant plant. The plant may be a man-made plant. Preferably, the transgene or mutation in the sequence of interest is located in germline or germline progenitor cells and/or tissue and/or plant part for clonal propagation of a recalcitrant plant. Therefore preferably, the plant comprises at least germline or germline progenitor cells of the recalcitrant plant and/or a tissue and/or a plant part for clonal propagation of the recalcitrant plant and preferably comprises the transgene or mutation in a sequence of interest. Preferably, the plant of the invention is not, or is not exclusively, obtained by an essentially biological process. The plant of the invention preferably differs at least from a plant occurring in nature, in that it contains at least one transgene or mutation in one sequence of interest. The transgene or mutation in the sequence of interest is preferably located in at least the germline or germline progenitor cells and/or tissue and/or plant part for clonal propagation of the plant. The transgene or mutation in the sequence of interest is preferably located in at least the L2-shoot meristem layer. The transgene or mutation in the sequence of interest is preferably present in at least one of the pollen and egg of the plant.

The plant preferably comprises at least germline or germline progenitor cells and/or tissues and/or plant parts for clonal propagation of a recalcitrant plant. The plant preferably comprise at least the L2-shoot meristem layer of a recalcitrant plant. The plant obtainable from the method of the invention is preferably a recalcitrant plant, preferably comprising a transgene or mutation in a sequence of interest.

The invention further pertains to offspring or seed from the plant or periclinal chimera as defined herein. The offspring may be produced by sexual or a-sexual (vegetative) propagation. The offspring preferably comprises a transgene or mutation in a sequence of interest as defined herein. The integument of the seed may have a different genotype than the embryo. Preferably, the genotype of integument is from the regenerative plant and the genotype of the embryo is from the recalcitrant plant.

The invention also concerns a plant part or plant product derived from a plant obtained from the method of the invention, preferably of step (c), (d) or (e) of the method of the invention. Optionally, said plant part or plant product is characterized in that it comprises genetic material originating from both the recalcitrant plant as well as the regenerative plant. Preferably, said plant part or plant product comprises cells or tissues or genetic material derived from the recalcitrant plant. Optionally, said plant part or plant product is free or substantially free of cells or tissues or genetic material that is derived from the regenerative plant. Optionally, said plant part or plant product consist of cells or tissues or plant material are characterized in that it comprises the genotype of the recalcitrant plant. Optionally, the plant part or plant product is characterized in that it comprises a transgene or mutation in a sequence of interest. Such genetic material may be genomic DNA or fragments of genomic DNA. Such genetic material may be mitochondrial DNA or fragments of mitochondrial DNA. Such hereditary material may be chloroplast DNA or fragments of chloroplast DNA.

The plant part may be propagating or non-propagating material. All patent and literature references cited in the present specification are hereby incorporated by reference in their entirety.

Optionally, the cells in step (a) of the method of the invention are contacted using tissue grafting methods. The inventors noticed that a grafting method making use of steel pins for fixation (preferably sterile steel pins), i.e. by inserting said steel pins in the centre of the stock and scion as exemplified herein (e.g. see FIG. 1), is surprisingly effective. Such grafting method using steel pins is particular useful when using seedling material, i.e. young plant material, preferably seedling material of between 1-4, or between 1-3 weeks after sowing, preferably seedling material of about 2 weeks after sowing, preferably seedling material of between 0.1 and 1 mm, between 0.1 and 0.75 mm, between 0.1 and 0.5 mm, or between 0.1 and 0.25 mm, preferably seedling material is used just after development of the first true leaves. The grafting method may be used in step (a) of the method of the invention, but also for producing a periclinal chimera. A grafting method wherein a steel pin is used for fixation may be, but is not limited to, a method as described herein in case said periclinal chimera comprises germline or germline progenitor cells and/or tissues and/or plant parts for clonal propagation of a recalcitrant plant. Optionally, said grafting method, e.g. for producing a periclinal chimera, finds broader application. Such method would comprise the same steps as the method of generating and selecting a shoot of a plant optionally comprising germline or germline progenitor cells and/or plant parts for clonal propagation of a recalcitrant plant as defined herein, albeit that the plants to be grafted in step (a) are not limited to being recalcitrant and/or regenerative, but only require both plants to have a different genotype, and the resulting shoot to be selected in step (d) is a periclinal chimera, not necessarily comprising germline or germline progenitor cells and/or tissues and/or plant parts for clonal propagation of a recalcitrant plant. Optionally a plant is grown from such periclinal chimera, which may find applications such as, but not limited to, specified in WO2018/115395 and/or WO2018/115396. Such method and resulting periclinal chimera can be considered as a further invention provided herein.

### BRIEF DESCRIPTION OF THE DRAWINGS

FIGS. 1A-1C. Exemplary representation of an embodiment of the invention. (FIG. 1A) Schematic representation of an in vitro propagated Bintje potato grafted as scion (2) onto a tomato rootstock (3), showing the optional steel pin (1) for fixation; (FIG. 1B) After healing, the graft union was decapitated at the graft healing. The cut site is indicated (4); (FIG. 1C) Decapitated grafts were left for regeneration of shoots (5).

FIGS. 2A-2C. Co-regenerated plants: pure tomato (FIG. 2A); Periclinal chimera of Bintje and tomato (FIG. 2B); pure Bintje (FIG. 2C).

FIGS. 3A-3D. Protoplast culture of Chicory intybus in K1Cg at 28° C. in the dark. FIG. 3A) Protoplasts obtained from overnight enzymatic digestion of the leaves of chicory, FIG. 3B) initial division seen after 4d of culture of chicory protoplasts in K1Cg medium, FIG. 3C) micro-colonies seen after 7d of culture and FIG. 3D) after 11d of culture.

FIGS. 4A-4D. Protoplast culture of Taraxacum brevicorniculatum in K1Cg medium in the dark at 28° C. FIG. 4A) Protoplasts obtained from overnight enzymatic digestion of Taraxacum leaves, FIG. 4B) vacuolated protoplasts showing no signs of division after 3 d of culture in K1Cg medium, FIG. 4C) and FIG. 4D) showing vacuolated protoplasts and debris of protoplasts after 10d and 17d after culture.

FIGS. 5A-5D. Regeneration of plantlets from protoplast mixtures of chicory and taraxacum. FIG. 5A) and FIG. 5B) show two different occurrences of mixed calli giving rise to a chicory and a taraxacum plantlet (dashed circle) which can be recognized distinctly by their varied phenotypes. FIG. 5C) Regeneration of chicory plantlets from control chicory calli and FIG. 5D) yellow calli picked from co-culture of mixed protoplasts giving rise to Taraxacum brevicorniculatum plantlets.

FIG. 6. PCR reaction showing the two taraxacum specific bands (794 bp) and the chicory specific bands (429 bp) in the Taraxacum control sample (lane 1), the Chicory control sample (lane 2) and in the leaf material samples from the co-regenerants having the taraxacum phenotype (lanes 3, 5, 7 and 8) or the chicory like phenotypes (lane 4 and 6).

FIGS. 7A-7C. Development of a chimeras of Maor grafted on C. baccatum. (FIG. 7A); The left side of plant 1 showed a C. baccatum phenotype with C. annuum trichomes, while the right side had a pure C. baccatum phenotype. (FIG. 7B); plant number 2 had a similar split phenotype, with the left side having a pure C. annuum phenotype, including anthocyanin patches and the right side having a pure C. baccatum phenotype. (FIG. 7C); Plant number 7 having C. annuum trichome morphology, with C. baccatum growth characteristics.

FIG. 8. Caps assay results are presented of these two leaf samples (3^{rd} and 4^{th} leave). The results show that plant 1, 2 and 7 are chimeras, with plants 3, 4, 5 and 6 being pure C. annuum.

FIG. 9. Left: RUBY and vYFP protoplasts grafted in a 170 micron Nitex mesh. Right: protoplast grafts from left after flushing of the mesh into 9M medium. Clumps of cells now are freely floating in liquid medium. Note the aggregation of both cell types together due to addition of β-D-Galactosyl Yariv.

FIGS. 10A-10C. (FIG. 10A) Abundant shoot regeneration from callus at the tip of a wild type tomato hypocotyl. (FIG. 10B) gob mutant hypocotyls form little callus with an occasional aberrant leaf, but no functional shoot meristems. (FIG. 10C) CAPS marker analysis of a graft hybrid plant (chi), co-regenerated from WT-gob graft junctions.

### EXAMPLE 1

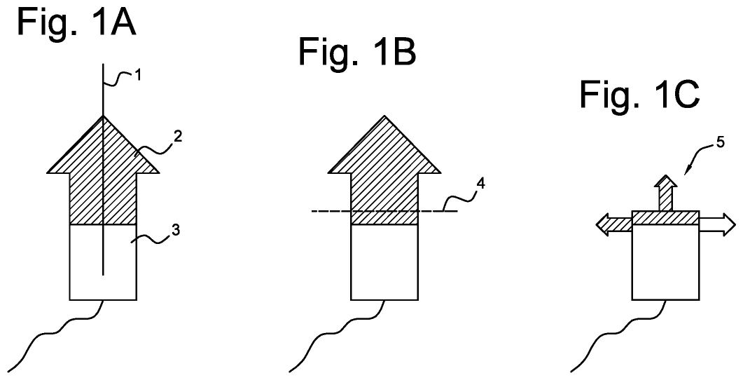

Potato propagation (Solanum tuberosum cultivar Bintje, originating from The Netherlands) was performed by rooting single node cuttings (an internode with an axillary bud) on hormone-free solid MS20 medium Murashige Skoog incl. vitamins, 20 g/L sucrose, no hormones). Various genotypes of tomato originating from The Netherlands (i.e. hybrids of Solanum lycopersicum cultivar Money Maker with Solanum habrochaites accessions LA1392 or PI27826, Solanum pennellii accession LA716, or Solanum lycopersicum accession LA3579) were sown on sterile filter paper drenched in sterile tap water, and placed on MS20 after root emergence. Grafting was done using a tomato seedling hypocotyl (˜two weeks after sowing, just after the development of its first true leaves) in MS20 as stock and potato nodal shoot tip of a young single node cutting as scion and using a sterile steel pin (0.15 mm diameter) inserted through the centre of the stock and scion for fixation, as shown in FIG. 1a. Grafts were placed on MS20 medium in a sterile tissue culture container, at 23° C. and a 16/8 hrs light/dark regime.

A total number of 251 grafts were healed for 6-9 days, after which they were decapitated as shown in FIG. 1b, leaving a thin layer of potato (˜1 mm) on the tomato rootstock, and were left to regenerate shoots in vertical position in MS20 as shown in FIG. 1c, without the addition of any hormones.

While tetraploid clonally propagated potato like Bintje does not regenerate in the absence of externally supplied plant hormones, tomato regenerates very quickly from severed hypocotyls on MS20, with the first signs of shoot emergence visible after 6-12 days. By scoring morphological features (1) leaf shape, and (2) trichomes, three potato-tissue containing shoots were identified within 7-14 days from the total of 251 grafts. This amount increased to a total of ten potato-tissue containing shoots in 1 months after grafting, across the different graft combinations with of Bintje with different tomato genotypes. It therefore seems that potato tissue regeneration is irrespective of the tomato genotype used. Of these 10 shoots, 3 were pure potato, 6 were mericlinal chimeras and 1 was a periclinal chimera. Regeneration of these shoots occurred at the interface of the graft (the junction). Therefore, the inventors concluded that potato was regenerated under the influence of co-present tomato cells at the graft junction.

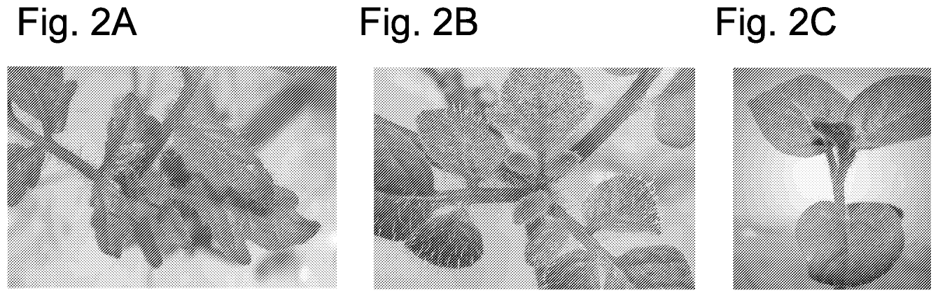

FIG. 2 shows pictures of the pure tomato, the periclinal chimera, and the Bintje regenerant.

### EXAMPLE 2

Seeds of Chicorium intybus L. (chicory Roodlof Indigo, Vreeken's Zaden, The Netherlands), herein further indicated as chicory, were surface sterilized with 70% ethanol for 30 sec and then sterilized with 2% bleach (sodium hypochloride solution) for 15 min and rinsed 3 times with sterile MQ 15 min at each rinse and sown in pots of MS (Murashige and Skoog, 1962) 20 hormone free medium at the pH of 5.8 and 0.8% agar in the dark.

Seeds of Taraxacum brevicorniculatum (provided by Dr. Jan Kirschner, Institute of Botany, Průhonice, Czech Republic), herein further indicated as taraxacum, were first rinsed with 70% ethanol for 30 sec and then sterilized with 1% bleach for 15 min. The seeds were then rinsed with sterile MQ water 4 times. The seeds were placed on a rolling shaker during the whole process of sterilization. Then the seeds were sown in MS20 supplemented with cefotaxime and vancomycin at pH 5.8 and 0.2% agar in the dark. Once the seeds germinated they were placed in pots of MS20 hormone free medium to develop into plants under normal light conditions (16 h light and 8 h darkness).

Chicory intybus is highly regenerative and Taraxacum brevicorniculatum is recalcitrant.

### Protoplast Isolation

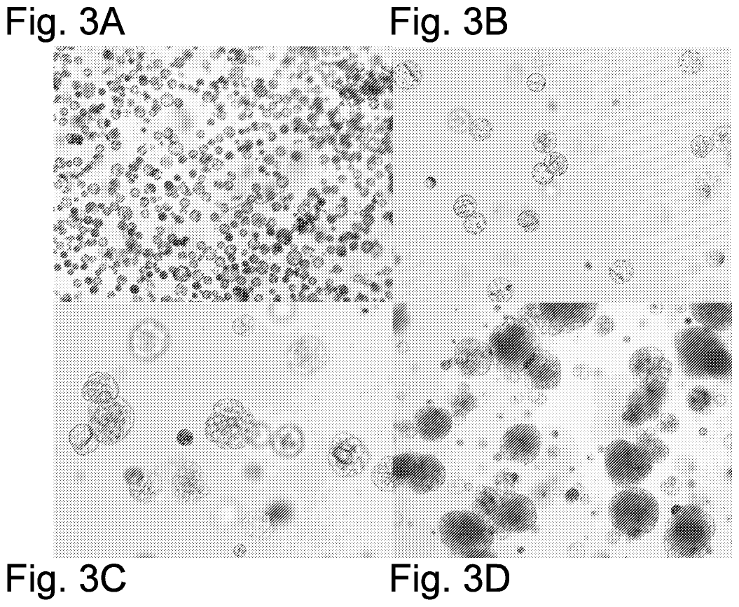

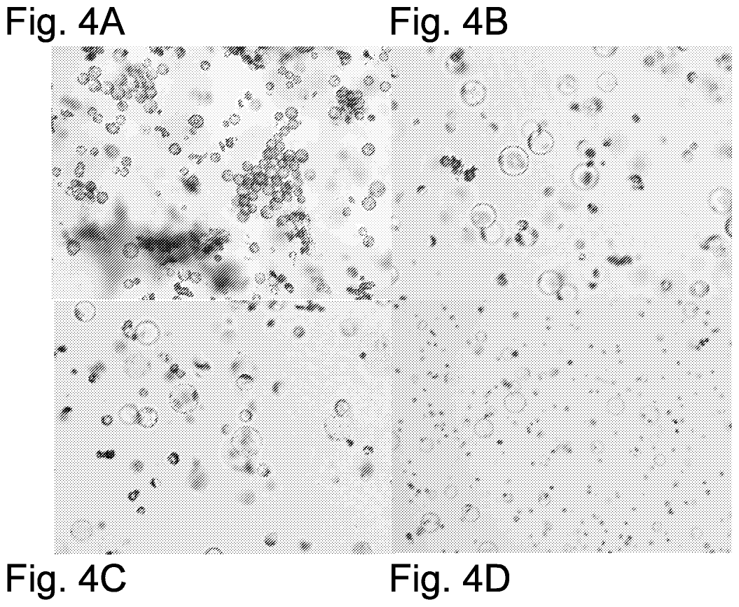

Once the plants were 4-6 weeks old, chicory and taraxacum protoplasts were isolated from leaf material of chicory and taraxacum plants, respectively, as follows. The 3 youngest leaves of the plants were cut along the venation of the leaves with sterile scalpels (sterile #10 surgical blades, Swaan Morton) in CPW9M (27 mg/L KH₂PO₄, 100 mg/L KNO₃, 200 mg CaCl₂·2H₂O, 512 mg MgSO₄·7H₂O, 0.16 μg KI, 0.39 ng CuSO₄·H₂O, 9% mannitol, 2.5 mg/L Fe(SO₄)₃·6H₂O and 580 mg/L MES at pH 5.8) and plasmolysed by incubation in 25 mL CPW9M in petri-dishes (Greiner 664161) for 30 min, after which the CPW9M was replaced by 25 mL of enzyme solution (1% Cellulase Onozuka RS (C8003, Duchefa), 0.2% Macerozyme R-10 (M8002, Duchefa) prepared in CPW9M at pH 5.8 supplemented with 4.44 μM BAP (6-Benzylaminopurine), 10.74 μM NAA (1-Naphthaleneacetic acid) and 0.9 μM 2,4-D (Dicloro-phenoxyacetic acid)) and placed in the dark at 25° C. for overnight digestion (16-17h; see FIG. 3A and 4A, respectively). After digestion, chicory and taraxacum protoplasts were purified using 12.5% (w/v) sucrose solution.

### Chicory Protoplast Inactivation

The purified chicory protoplasts used for co-regeneration were re-suspended in CPW9M and treated with 10 mM lodoacetamide (IOA, Sigma) for 30 min. IOA acts as an irreversible inhibitor of the mitotic-spindle assembly at the prophase of mitosis, thus impeding the cell division (Varotto et al. 2001). After IOA treatment the protoplasts were rinsed twice with CPW9M.

### Taraxacum Protoplast Staining

The purified taraxacum protoplasts were also re-suspended in CPW9M. As taraxacum protoplasts do not divide in K1Cg and K5Cg, which is the chicory medium of choice, there is no need to treat them with IOA. Instead, the taraxacum protoplasts were treated with fluorescein diacetate (FDA, Widholm 1972) for 5 min for visualisation purposes.

### PEG Treatment

The IOA treated chicory and FDA treated taraxacum protoplasts were admixed at a ratio of either 1:1, 1:2 or 1:3 at a total protoplast density of 1×10⁶ per mL. In order to bring the protoplasts in close proximity with each other, each of these mixtures were treated with a PEG (Polyethylene glycol) 3350 MW solution (30 g of PEG 3350 MW, 150 mg CaCl₂2H₂O, 10 mg KH₂PO4 in a final volume of 100 mL H₂O at pH 5.5) and subsequently with a neutralizing solution (735 mg CaCl₂·2H₂O, 375 mg of glycine, 8 g of mannitol in a final volume of 100 mL H₂O, at pH 10.5) using either Method 1 or Method 2.

### Method 1

Per experiment, 1 mL of the protoplast mixture was pipetted gently in 4 drops on a 6 cm petri-dish (Greiner 628102, SigmaAldrich). The same volume of PEG 3350 MW solution was added drop wise around the protoplasts mixture gently. The protoplast mixture and PEG solution were mixed very carefully and incubated for 30 min at room temperature. Subsequently, 10 mL of the neutralizing solution was added was to the mixture. Then, the mixture was centrifuged at 800 rpm for 5 min and rinsed with 9M (9% (w/v) mannitol, 140 mg/L CaCl₂·H₂O, 580 mg/L MES at pH 5.8) twice before embedding them in alginate discs as further specified below.

### Method 2

Per experiment, 200 μL of the protoplast mixture was placed in a petri dish and 3 volumes of the above indicated PEG 3350MW solution was added dropwise to the protoplast mixture. After 1 min of incubation, 700 μL of the above indicated neutralizing solution was added twice at 1 min intervals. Five min later, 2 mL of CPW9M was added 3 times at 5 min intervals. After incubation for 10-15 min at room temperature, the fusion mixture was centrifuged for 10 min at 800 rpm and rinsed with 9M twice before embedding them in alginate discs as further specified below.

### Protoplast Culturing

Protoplast were embedded in alginate discs by gently admixing protoplast suspension in 9M 1:1 with a 1.6% Na-alginate (S1320, Duchefa) solution comprising 9% (w/v) mannitol to obtain a final protoplast density of 0.25×10⁶ per mL, and subsequently gently spreading 1 mL of this mixture onto calcium agar plates. This was performed for the protoplast mixtures and pure (i.e. non-admixed) chicory and taraxacum protoplasts as control. Alginate discs were allowed to polymerize by incubation at room temperature for 45 min. The discs were transferred into 6 cm petri-dishes (Greiner 62810, Sigma-Aldrich) with 4 mL of K1Cg medium supplemented with NAA and BAP (1.9 g/L KNO₃, 600 mg/L CaCl₂·2H₂O, 300 mg/L MgSO₄·7H₂O, 170 mg/L KH₂PO₄, 300 mg/L KCl, 750 μg/L KI, 3 mg/L H₃BO₃, 10 mg/L MnSO₄·H₂O, 2 mg/L ZnSO₄·7H₂O, 250 μg/L Na₂MoO₄·2H₂O, 25 μg/L CuSO₄·5H₂O, 25 μ/L CoCl₂·6H₂O, 20 mg/L Na-pyruvate, 40 mg/L citric acid, 40 mg/L malic acid, 40 mg/L fumaric acid and 100 mg/L myo-inositol, supplemented with 2.5 mg/L sucrose, 2.5 mg/L fructose, 2.5 mg/L ribose, 2.5 mg/L xylose, 2.5 mg/L mannose, 2.5 mg/L rhamnose, 2.5 mg/L cellobiose, 2.5 mg/L sorbitol, 2.5 mg/L mannitol, 1% (v/v) Kao and Michayluk vitamin solution (K3129, Sigma-Aldrich) 2% (v/v) Coconut water (C5915, Sigma-Aldrich), 27.8 mg/L FeSO₄·7H₂O, 37.7 mg/L Na₂EDTA·2H₂O, 68.4 g/L glucose, 300 mg/L glutamine, 2 mg/L NAA, 1 mg/L BAP, at pH 5.8) in the dark at 28° C.

After 1 week the K1Cg medium was removed and was replaced by 4 mL K5CgK medium (600 mg/L CaCl₂·2H₂O, 300 mg/L MgSO₄·7H₂O, 170 mg/L KH₂PO₄, 300 mg/L KCl, 750 μg/L KI, 3 mg/L H₃BO₃, 10 mg/L MnSO₄·H₂O, 2 mg/L ZnSO₄·7H₂O, 250 μg/L Na₂MoO₄·2H₂O, 25 μg/L CuSO₄·5H₂O, 25 μ/L CoCl₂·6H₂O, 20 mg/L Na-pyruvate, 40 mg/L citric acid, 40 mg/L malic acid, 40 mg/L fumaric acid and 100 mg/L myo-inositol, supplemented with 2.5 mg/L sucrose, 2.5 mg/L fructose, 2.5 mg/L ribose, 2.5 mg/L xylose, 2.5 mg/L mannose, 2.5 mg/L rhamnose, 2.5 mg/L cellobiose, 2.5 mg/L sorbitol, 2.5 mg/L mannitol, 1% (v/v) Kao and Michayluk vitamin solution (K3129, Sigma-Aldrich) 2% (v/v) Coconut water (C5915, Sigma-Aldrich), 27.8 mg/L FeSO₄·7H₂O, 37.7 mg/L Na₂EDTA·2H₂O, 52.5 g/L glucose, 600 mg/L glutamine, 750 mg/L KCl, 0.5 mg/L NAA, 0.5 mg/L BAP, at pH 5.8) and the protoplasts were cultured in the dark at 28° C. After 2 weeks of culture in K5CgK, the alginate discs were cut into strips of ˜5 mm strips and transferred onto solid B5g-10 medium supplemented with 2.69 μM I NAA and 2.22 μM BAP and 1% sea plaque agarose (S1202, Duchefa).

Once the micro-colonies formed, micro-calli of 1-3 mm size (2-3weeks) they were hand-picked with fine tweezers and 50 micro-calli were placed onto square petri-dishes (Greiner 688102, SigmaAldrich) with MS10 medium supplemented with 1.43 μM IAA (Indoleacetic acid, Duchefa) and 1.11 μM BAP. After 3 weeks the calli (initial signs of regeneration) were then transferred to SH (Schenk & Hildebrandt medium, Duchefa) 10 medium with the same hormone combinations. Once shoot like structures were observed they were transferred into pots (OS60 pots, Duchefa) of SH10 with 1.43 μM IAA and 1.11 μM BAP. The developing shoots were then transferred to square pots (Aarts plastics) with SH10 hormone free medium and they were cultured at 25° C. with 16 h light and 8 h dark conditions. The regenerated plants were genotyped with specific primers for chicory and taraxacum.

### Results

### Co-Regeneration

Pure chicory protoplasts started initial division during the first week of protoplast culture in K1Cg medium supplemented with NAA and BAP 3-4 days after isolation (FIG. 3B) and formed micro-colonies after 4 days (FIG. 3C). Chicory colonies were seen after 11 days of culture (FIG. 3D).

Pure taraxacum protoplasts were recalcitrant and did not divide in K1Cg medium as shown in FIG. 4B-D. There were no signs of initial division after 3 days of culture. After 10 and 17d days of culture, the taraxacum protoplasts became vacuolated and showed no viable signs.

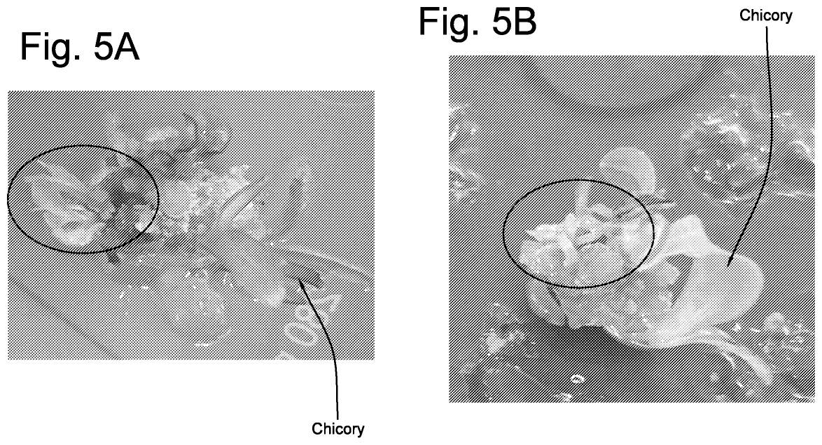

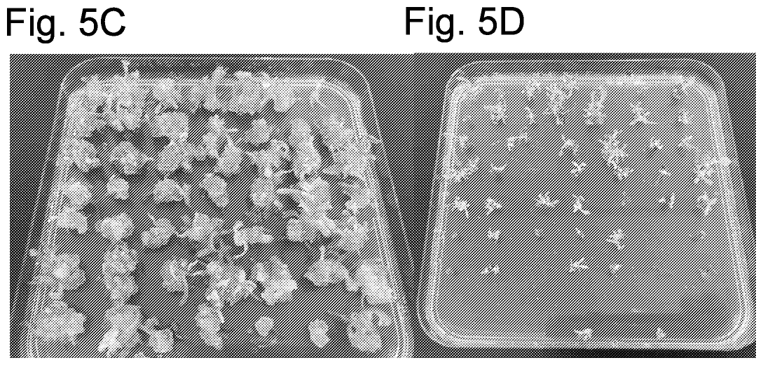

Upon co-culture of chicory and taraxacum protoplasts admixed at 1:1 and 1:2, yellowish calli were formed after 8-10 weeks of protoplast culture after isolation. These calli were clearly different in phenotype from the whitish watery calli of chicory. These yellowish calli appeared in experiments using protoplasts admixtures at chicory and taraxacum protoplasts ratios of 1:1 and 1:2 and in both PEG treatment protocols (Method 1 and Method 2). Once these calli started showing signs of regeneration, it was observed that two distinct phenotypes appeared within each single callus (FIG. 5A and B).

The plantlets derived from whitish chicory calli had broader leaves like that of chicory whereas the yellowish calli gave rise to thin leaves like that of taraxacum (FIG. 5 C and D).

The leaves of the regenerated plantlets were sampled and PCR was conducted on DNA samples of these leaves by 35 cycles at an annealing temperature of 57° C. using a chicory specific primer set or a taraxacum specific primers set, respectively. The chicory specific forward primer had the sequence of 5′-CAGACACAATGGTAGATGATGG-3′ (SEQ ID NO: 10) and chicory specific reversed primer had the sequence of 5′-CTTCATCGCCATGCCCAGAAG-3′ (SEQ ID NO: 11) giving rise to a 429 bp band. The forward primer of the taraxacum primer set had the sequence of 5′-TAAGAAACCGAAGCAAACTC-3′ (SEQ ID NO: 12) and reversed primer of the taraxacum primer had the sequence of 5′- GCGCTTTCTACAATCTTACA-3′ (SEQ ID NO: 13) giving rise to a 794 bp band.

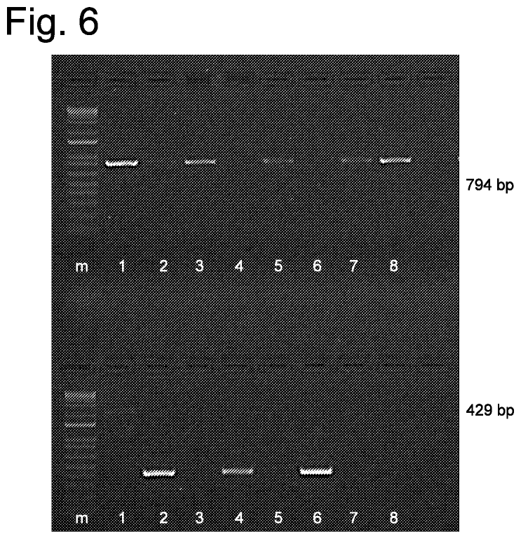

It was observed that the plantlets from the co-cultures having the Taraxacum like phenotype gave only the taraxacum specific band whereas the chicory like phenotype gave only the chicory specific bands (FIG. 6).

This is proof of concept that two widely different species, i.e. Chicory intybus (highly regenerative) and Taraxacum brevicorniculatum (recalcitrant), co-regenerated from a protoplast co-culture using PEG 3350MW.

### EXAMPLE 3

Maor pepper (Capsicum annuum; Israel) is known for being a recalcitrant plant, showing no shoot meristem regeneration upon wounding and/or tissue culture. A total of 201 grafts of a Maor cotyledonary node as scion on a C. baccatum (cultivar rainforest, Vreeken's Zaden, The Netherlands) seedling hypocotyl as stock were made and decapitated according to the same method as described in Example 1, with the exception that grafts were placed on MS10 containing 1% sucrose instead of MS20 containing 2% sucrose. Like in Example 1, after decapitation, the stumps were left to regenerate without the addition of any hormones.

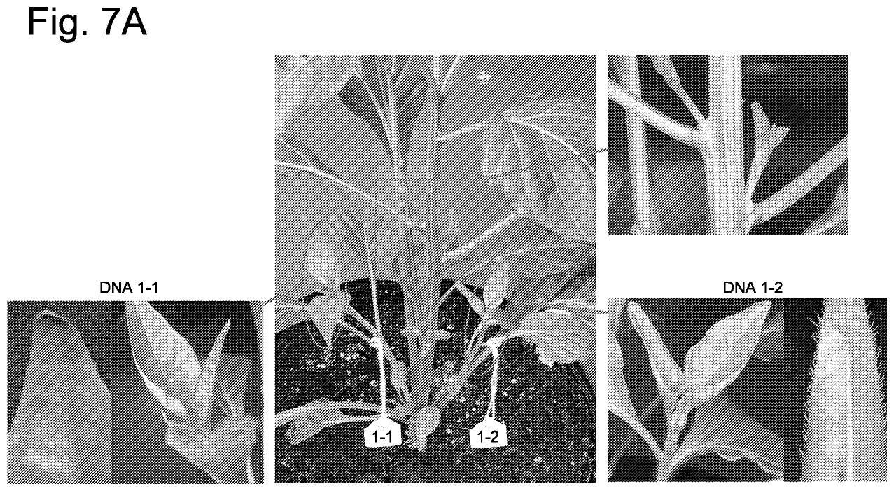

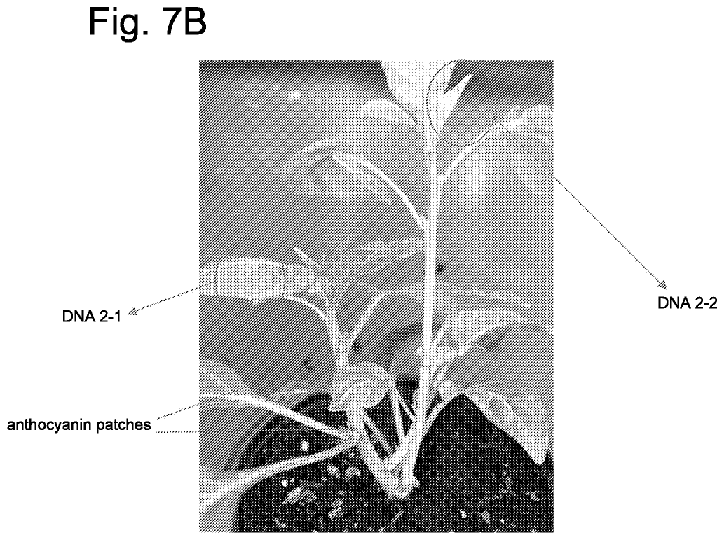

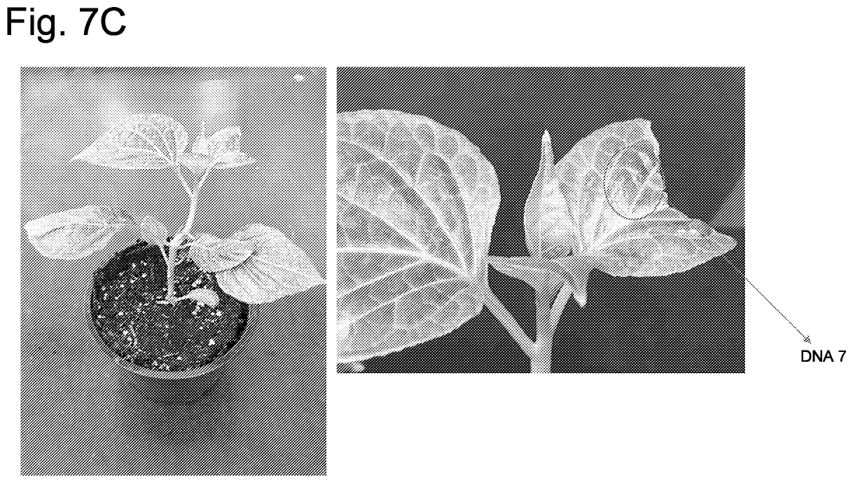

As judged by trichome morphology, two Maor-tissue containing leaves (mericlinal chimeras) and five plants with pure Maor leaves and shoots were identified within 7-10 days from the total of 201 grafts. The left side of chimera 1 showed an overall C. baccatum phenotype, with C. annuum trichomes and is likely a periclinal chimera. The right side showed a pure C. baccatum phenotype. The left side was marked 1-1 and the right side was marked 1-2 for further analysis (FIG. 7A). Chimera 2 had a similar phenotype, where the left side phenotypically appeared as pure C. annuum and the right side had an appearance of pure C. baccatum (FIG. 7B). After transfer to the greenhouse, plant number 7, which was identified as a pure C. annuum based on trichome morphology, showed a C. baccatum growth phenotype and was therefore identified as possibly being a periclinal chimera (FIG. 7C).

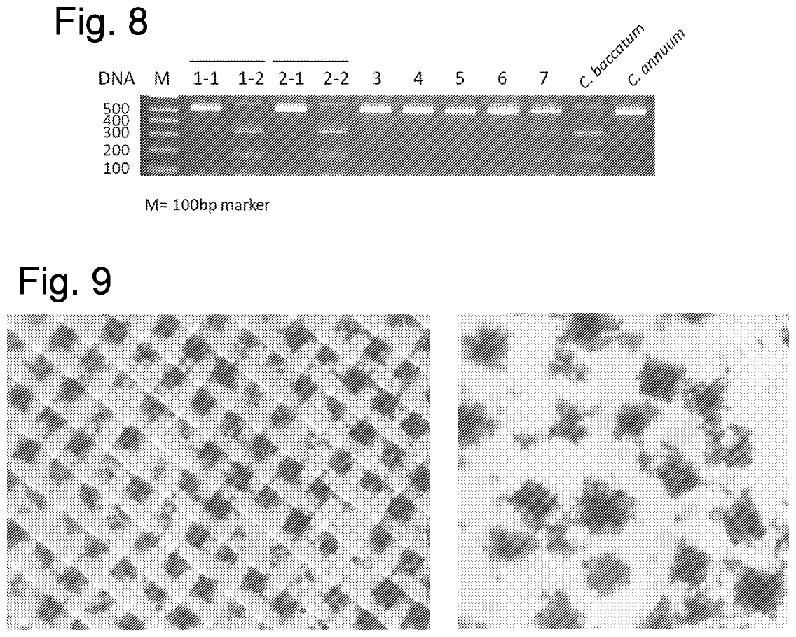

With a CAPS marker assay with a forward primer having the sequence 5′ atactaatttccacccaacaacgt 3′ (SEQ ID NO: 14) and a reversed primer having the sequence 5′ tctcaacattaaacatgtcgccac 3′ (SEQ ID NO: 15), PCR amplification was performed on samples of these seven plants. In particular, using these primers, PCR amplification was performed on DNA isolated from leaf samples by 34 cycles and an anneal temperature of 55° C. The 515 bp products were incubated for 1 hour with EcoRV. Because of the EcoRV recognition site present in the amplified genomic sequence of C. baccatum, that is absent in the amplified genomic sequence of Maor, EcoRV incubation of amplicons from Maor resulted in 515 bp fragments, whereas EcoRV incubation of amplicons from Maor resulted in fragments of 348 bp and 167 bp. FIG. 8 shows that samples taken from each plant support the phenotype analysis, with plants 1, 2 and 7 being chimeras and plants 3, 4, 5 and 6 being pure C. annuum.

### EXAMPLE 4

Tobacco (Nicotiana benthamiana; Herbalistics, Australia) protoplast grafting and co-regeneration was performed using two transgenic tobacco cell lines. One cell line carrying venus yellow fluorescent protein under control of a 35S promotor (35S::vYFP) and a second cell line carrying cytoplasmic RUBY (He et al. Horticulture Research (2020) 7:152) under control of a 35S promotor (35S::RUBY). To prepare the grafting in vitro shoot cultures of tobacco, grown on MS20 medium without hormones with an 18/8H photoperiod at 25° C. and 60%-70% relative humidity were used.

Tobacco protoplasts were isolated from these shoot cultures. Young fresh leaves form the tobacco plants were collected upside down in a square Petri dish containing 5 mL of CPW9M (27 mg/L KH₂PO₄, 100 mg/L KNO₃, 200 mg/L CaCl₂·2H₂O, 512 mg/L MgSO₄·7H₂O, 0.16 μg/L KI, 0.39 ng/L CuSO₄·H₂O, 9% mannitol, 2.5 mg/L Fe(SO₄)₃·6H₂O and 580 mg/L MES at pH 5.8). The lower epidermis was sliced perpendicular to the main vein every millimeter, by carefully holding down the leave and using a fresh scalpel. The sliced leaves were transferred to a 15 cm Petri dish containing 15 mL of CPW9M. Once all the leaves were sliced, the sliced material was transferred to the 15 cm Petri dish, and 5 mL of Enzyme stock SR1 (0.75% Cellulase Onozuka RS (C8003, Duchefa), 0.5% Driselase (D8037, Sigma), 0.25% Macerozyme R-10 (M8002, Duchefa) prepared in CPW9M at pH 5.8) was added to the dish and gently swirled. The dish was covered with cling film and incubated at 25° C. for 18 hours. To release the protoplasts, the plate was continuously swirled.

Next the protoplasts were washed using the following steps. A stainless steel 50 μm sieve was pre-wetted with CPW9M. Without taking up large debris, the protoplast solution was pipetted up and transferred to the sieve. 25 mL of KC (2 g/L CaCl₂·2H₂O, 19 g/L KCl and 580 mg/L MES at pH 5.8) was added to the remaining debris to release more protoplasts and transferred carefully to the sieve. This step was repeated using 12.5 mL of KC and subsequently the sieve was rinsed with another 15.3 mL of KC. The flow through was distributed over centrifuge tubes and spun down for 5 min at 85×g. After removing the supernatant, 5 mL of CPW9M was added to the pellet and re-suspended. Two tubes were combined into one and the centrifuge step was repeated. After the supernatant was removed, the pellet was again re-suspended in 5 mL of CPW15S (CPW supplemented with 150 g/L sucrose). Subsequently, the pellet was overlaid with 2 mL of CPW9M, without mixing the two layers. The centrifuge step was repeated for 10 minutes and the protoplasts were collected from the interphase layer, without disturbing the sucrose layer. The final protoplast density was set to 2×10⁶ per mL in CPW9M.

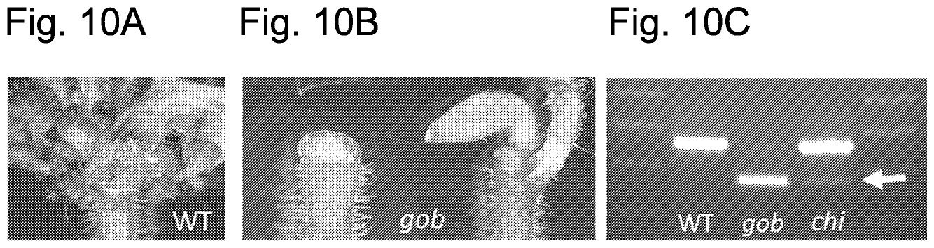

To graft the protoplasts we used sterile 2×2 cm pieces of precision mesh (Nitex #03-177/34) of 170 micron mesh opening and 220 micron thickness, which were dry and kept sterile until use. The precision mesh was applied on a K8P agar plate (600 mg/L CaCl₂·2H₂O, 300 mg/L MgSO₄·7H₂O, 170 mg/L KH₂PO₄, 300 mg/L KCl, 600 mg/L NH₄NO₃, 1.9 g/L KNO₃, 750 μg/L KI, 3 mg/L H₃BO₃, 10 mg/L MnSO₄·H₂O, 2 mg/L ZnSO₄·7H₂O, 250 μg/L Na₂MoO₄·2H₂O, 25 μg/L CuSO₄·5H₂O, 25 μ/L CoCl₂·6H₂O, 20 mg/L Na-pyruvate, 40 mg/L citric acid, 40 mg/L malic acid, 40 mg/L fumaric acid and 100 mg/L myo-inositol, supplemented with 2.5 mg/L sucrose, 2.5 mg/L fructose, 2.5 mg/L ribose, 2.5 mg/L xylose, 2.5 mg/L mannose, 2.5 mg/L rhamnose, 2.5 mg/L cellobiose, 2.5 mg/L sorbitol, 2.5 mg/L mannitol, 1% (v/v) Kao and Michayluk vitamin solution (K3129, Sigma-Aldrich) 2% (v/v) Coconut water (C5915, Sigma-Aldrich), 27.8 mg/L FeSO₄·7H₂O, 37.7 mg/L Na₂EDTA·2H₂O, 68.4 g/L glucose, 3 mg/L NAA, 1 mg/L BAP, at pH 5.8) without trapping air under the mesh. A 50 μL aliquot of the protoplasts of each of the two cell types was mixed in a 2 mL Eppendorf tube by using a wide tip pipette and 2 uL of β-D-Galactosyl Yariv (2 mg/mL, Biosupplies Australia Pty. Ltd., Cat. No:100-8A) solution was added. Yariv ensures the aggregation of the protoplasts, enabling co-regeneration. Without the Yariv solution no bonding between the protoplasts occurred (data not shown). The mixture was immediately spotted on the mesh in droplets, making sure the pores of the mesh are saturated with cells and left to stand for 5 minutes so no dome shaped droplets were visible. The spots were carefully overlaid with a K8P 1,6% agar cover slip, while pushing aside any excess cells from the mesh, and left to agglutinate for 25 minutes. The agar slide was removed from the mesh as well as the mesh from the agar plate. The mesh was rinsed with 1 mL of 9M medium (90 g/L Mannitol, 140 mg/L CaCl₂·2H₂O, pH 5.8) into a 5 cm Petri dish (see FIG. 10). To embed the protoplasts, 1 mL alginate solution (1.6% Na-alginate (S1320, Duchefa), 140 mg/l CaCl₂·2H₂O, 9% (w/v) mannitol) was added to the 1 mL of 9M+agglutinate suspension and mixed gently. The resulting mixture was dispersed on a Ca-Agar plate (72.50 g/L Mannitol, 7.35 g/L CaCl₂·2H₂O, 0.8% micro-agar, pH 5.8) and left for 1 hour. After 1 hour, the disc was transferred to 4 mL of K8P culture medium, sealed and incubated for 5 days at 28° C. in the dark.

To form calli, the K8p medium was replaced after the 5 days of incubation with 0.5 MS+2% sucrose+6% mannitol+0.03 mg/L NAA+0.1 mg/L BAP and incubated for 7 days. The alginate was dissolved using NaCitrate (14.7 g/L Tri Na Citrate·2H₂O, 36.4 g/L Mannitol at pH 6.5) and passed through a 160 micron sterile mesh (Nitex) to select for larger colonies. The colonies were embedded in alginate as described above and fresh 0.5 MS+2% Sucrose+6% Mannitol+0.03 mg/L NAA+0.1 mg/l BAP was added. The sealed plate was incubated in the dark at 25° C. After 5 days the medium was replaced with 0.5 MS+2% Sucrose+3% Mannitol+0.03 mg/L NAA+0.1 mg/L BAP and incubated as before. The medium was replaced with 0.5 MS+2% Sucrose+0.25 mg/L zeatin and incubated at 25° C. in the light. Every 3 weeks it was transferred to fresh medium until regeneration appeared. The disc or calli were transferred to a Petri dish with 0.5 MS+2% Sucrose+0.25 mg/L zeatin+0.8% micro-agar and let shoots develop for screening of desired type of regenerants.

In several independent experiments chimeric leaves developed from the selected and handpicked calli (in 10 out of 96 picked calli). These chimeric calli stood out in having red+green organs in a full green or red background. A fraction of these chimeras developed in mericlinal chimeras, from which periclinal chimeras were developed using axillary buds.

### EXAMPLE 5

Co-regeneration was also tested in tomato (RZ52201, Rijk Zwaan, The Netherlands) by grafting a regenerative wild type (WT) to a non-regenerative mutant in the goblet gene (gob; Berger Y. et al. (2009) The NAC-domain transcription factor GOBLET specifies leaflet boundaries in compound tomato leaves. Development 136 (5):823-832). Homozygous gob null mutants (gob-1, Berger et al. 2009) fail to (re-)generate shoots by defective specification/maintenance of stem cells (FIGS. 10A and 10B). Shoots regenerated from graft junctions formed by grafting gob null mutant scions on WT rootstocks (n=79 grafts) and subsequently culturing slices of these graft junctions. All were WT except for one stable (periclinal) chimera, in which the gob null formed a functionally normal epidermal L1 layer (FIG. 10C). Thus, stable stem cell identity was imparted onto gob cells by joint pattern formation during adventitious shoot formation, i.e. by co-regeneration.

1. A method of producing a shoot of a plant, comprising:
(a) contacting a cell of a recalcitrant plant with a cell of a regenerative plant, wherein the cell of the regenerative plant shows a higher regeneration efficiency than the cell of the recalcitrant plant under a condition that allows for shoot regeneration;
(b) allowing the contacted cells of (a) to form shoots under the condition that allows for shoot regeneration;
(c) selecting a shoot formed in (b), wherein at least part of the shoot comprises cells of the recalcitrant plant; and optionally
(d) growing a plant from the selected shoot of (c).

2. The method according to claim 1, wherein the part of the selected shoot is tissue comprising germline progenitor cells.

3. The method according to claim 1, comprising (d) growing a plant from the selected shoot of (c), and further comprising (e) obtaining seed or plant progeny of the plant grown in (d) by sexual propagation, optionally by selfing or backcrossing.

4. The method according to claim 1, comprising (d) growing a plant from the selected shoot of (c), and further comprising (e) obtaining progeny of the plant grown in (d) by vegetative propagation.

5. The method according to claim 1, wherein the cell of the recalcitrant plant and the cell of the regenerative plant of (a) are isolated cells.

6. The method according to claim 5, wherein the isolated cells are isolated protoplasts.

7. The method according to claim 1, wherein the cells contacted are exposed to a compound promoting aggregation of the cell membranes of the cells.

8. The method according to claim 7, wherein the compound promoting aggregation a plant cell and/or protoplast linking agent.

9. The method according to claim 8, wherein the linking agent is Yariv reagent.

10. The method according to claim 1, wherein the cell of the recalcitrant plant and the cell of the regenerative plant of (a) are comprised in a tissue.

11. The method according to claim 10, wherein the contacting (a) is performed by stock-scion grafting and allowing the graft junction to heal.

12. The method according to claim 11, wherein the allowing (b) comprises:
(i) generating a wound at or near the graft junction;
(ii) allowing callus to be formed at the wounded graft junction; and
(iii) allowing a shoot to grow from the callus.

13. The method according to claim 1, further comprising introducing in the cell of the recalcitrant plant of (a) or in a cell originating therefrom in the shoot formed in (b):
(i) a transgene; or
(ii) a mutation in a sequence of interest.

14. The method according to claim 13, wherein the introducing is prior to step (b).

15. The method according to claim 13, wherein the transgene or the mutation is in at least one of:
(i) the germline progenitor cell and/or a germline cell derived therefrom; and
(ii) a plant part of the plant grown in (d) used for vegetative propagation of the shoot formed in (b).

16. The method according to claim 13, wherein the mutation is introduced by programmed genome editing.

17. The method according to claim 16, wherein the mutation is introduced using a site-specific endonuclease.

18. The method according to claim 17, wherein the site-specific endonuclease is a CRISPR endonuclease.

19. A plant obtainable by the method of claim 13, wherein the plant comprises at least one of:
(i) a germline progenitor cell and/or a germline cell derived therefrom of the recalcitrant plant; and
(ii) a plant part for cional propagation of the recalcitrant plant,
wherein the germline progenitor cell, germline cell and/or a plant part comprises the transgene or the mutation in the sequence of interest.

20. The plant according to claim 19, comprising cells of the recalcitrant plant and cells of the regenerative plant.
